SEQUENCE LISTING (16 sequences)

SEQ ID NO: 1 - protein, 366 aa, Mus musculus
MTPGTQSPFFLLLLLTVLTATTAPKPATVVGSVVVQLTLAFREGTINVHDVETQFNQYKTEAASRYNLTISDVSVSDVPFPFSAQSGAGGGGGSGGGGSGGGGSKLEPRGPTIKPCPPCKCPGKSLDQSSTPGRMVSAINIPALEDKPCTDPFPSLLISTPRGWTIRLHLPSKDQGCTHDLPEPHSHMCGGGCERGPRCPDQLVCEQRGSTHSSDTNPRGLQQYSPGGQCPPHPAPGLDEWQGFRMRRQQQRPPSAHRENHLKTQRELQPDCMGAGMGIRIKVCVDSLLLQPPLYMFLPSQGQELHRYMSCLHQRKRLRNRSLPAWSQTSCLKTFTWSGPTTGKQSTTRTLNQSWTLMVLTSCTAS

SEQ ID NO: 2 - protein, 159 aa, Homo sapiens
GSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 3 - protein, 10 aa, Artificial Sequence
  Synthetic peptide
GGGGSGGGGS

SEQ ID NO: 4 - protein, 20 aa, Artificial Sequence
  Synthetic peptide
GGGGSGGGGSGGGGSGGGGS

SEQ ID NO: 5 - protein, 25 aa, Artificial Sequence
  Synthetic peptide
GGGGSGGGGSGGGGSGGGGSGGGGS

SEQ ID NO: 6 - protein, 10 aa, Artificial Sequence
  Synthetic peptide
EAAAKEAAAK

SEQ ID NO: 7 - protein, 15 aa, Artificial Sequence
  Synthetic peptide
EAAAKEAAAKEAAAK

SEQ ID NO: 8 - protein, 20 aa, Artificial Sequence
  Synthetic peptide
EAAAKEAAAKEAAAKEAAAK

SEQ ID NO: 9 - protein, 58 aa, Artificial Sequence
  Synthetic peptide
SVVVQLTLAFREGTINVHDVETQFNQYKTEAASRYNLTISDVSVSDVPFPFSAQSGAG

SEQ ID NO: 10 - protein, 15 aa, Artificial Sequence
  Synthetic peptide
GGGGSGGGGSGGGGS

SEQ ID NO: 11 - protein, 4 aa, Artificial Sequence
  Synthetic Peptide
GGGG

SEQ ID NO: 12 - protein, 62 aa, Artificial Sequence
  Synthetic Peptide
LSTGVSFFFLSFHISNLQFNSSLEDPSTDYYQELQRDISEMFLQIYKQGGFLGLSNIKFRPG

SEQ ID NO: 13 - protein, 388 aa, Artificial Sequence
  Synthetic Peptide
MYRMQLLSCIALSLALVTNSLSTGVSFFFLSFHISNLQFNSSLEDPSTDYYQELQRDISEMFLQIYKQGGFLGLSNIKFRPGGGGGSVVVQLTLAFREGTINVHDVETQFNQYKTEAASRYNLTISDVSVSDVPFPFSAQSGAGGGGGSGGGGSGGGGSRSPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 14 - DNA, 1167 bp, Artificial Sequence
  Synthetic nucleic acid
atgtacaggatgcaactcctgtcttgcattgcactaagtcttgcacttgtcacgaattcgttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggaggaggaggaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctgggggtggaggcggttcaggcggaggtggctctggcggtggcggatcgagatctcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatga

SEQ ID NO: 15 - protein, 402 aa, Artificial Sequence
  Synthetic peptide
MTPGTQSPFFLLLLLTVLTATTAPKPATVVLSTGVSFFFLSFHISNLQFNSSLEDPSTDYYQELQRDISEMFLQIYKQGGFLGLSNIKFRPGGGGGSVVVQLTLAFREGTINVHDVETQFNQYKTEAASRYNLTISDVSVSDVPFPFSAQSGAGGGGGSGGGGSGGGGSEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 16 - DNA, 1436 bp, Artificial Sequence
  Synthetic nucleic acid
atgacaccgggcacccagtctcctttcttcctgctgctgctcctcacagtgcttacagctaccacagcccctaaacccgcaacagttgttttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggaggaggaggaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctgggggtggaggcggttcaggcggaggtggctctggcggtggcggatcgaagcttgagccccgcggaccgacaatcaagccctgtcctccatgcaaatgcccaggtaagtcactagaccagagctccactcccgggagaatggtaagtgctataaacatccctgcactagaggataagccatgtacagatccatttccatctctcctcatcagcacctaacctcgagggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggctttcgcatgcgccgtcaacaacaaagacctcccagcgcccatcgagagaaccatctcaaaacccaaaggtgagagctgcagcctgactgcatgggggctgggatgggcataaggataaaggtctgtgtggacagccttctgcttcagccatgacctttgtatatgtttctaccctcacagggtcagtaagagctccacaggtatatgtcttgcctccaccagaggaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

(19) United States
(12) Patent Application Publication
(10) Pub. No.: US 2013/0101664 A1
(43) Pub. Date: Apr. 25, 2013

(54) MUC1 LIGAND TRAPS FOR USE IN TREATING CANCERS

(71) Applicants: Donald W. Kufe, Wellesley, MA (US); Surender Kharbanda, Natick, MA (US)

(72) Inventors: Donald W. Kufe, Wellesley, MA (US); Surender Kharbanda, Natick, MA (US)

(21) Appl. No.: 13/588,427

(22) Filed: Aug. 17, 2012

Related U.S. Application Data
(60) Provisional application No. 61/524,978, filed on Aug. 18, 2011.

(51) Int. Cl. C07K 14/47; A61K 45/06; A61K 39/395

(52) U.S. Cl. CPC C07K 14/4727; A61K 39/395; A61K 45/06
USPC 424/450; 435/375; 424/134.1; 424/490; 424/400; 530/387.3; 424/499

(57) ABSTRACT

The present invention is directed to improved compositions for the disruption of signaling through the external domain (ED) of MUC1, Ligand traps—molecules that include MUC1 ED sequences and immunoglobulin Fc domains—effectively “trap” molecules that interact with the native MUC1 ED. Given the involvement of MUC1 in a variety of disease states, disrupting the interaction of other molecules with MUC1 ED is useful in treating these disease, in particular cancer.

This application claims benefit of priority to U.S. Provisional Application Ser. No. 61/524,978, filed Aug. 18, 2011, the entire contents of which is hereby incorporated by reference.

This invention was made with government support under grant number CA 97098 awarded by The National Cancer Institute. The government has certain rights in the invention.

### BACKGROUND OF THE INVENTION

1. Field of the Invention

This invention relates to regulation of signaling involving MUC1. In particular, improved MUC1 “ligand traps” are used to disrupt signaling between the external domain of MUC1 and various binding partners. The disruption of signaling has significant consequences in MUC1-related disease states including cancers, particularly where MUC1 is overexpressed relative to amounts observed at normal levels.

2. Related Art

A. Cancer

Cancer is the second leading cause of death in the United States after heart disease. Analysis of the cancer incidence and mortality data from the National Cancer Institute estimates that over 1.4 million new cases will be diagnosed in 2008 and >650,000 will die from cancer in the United States. The development of new anti-cancer agents is focused on specific targets that contribute to tumor formation.

For example, the ErbB2 receptor is overexpressed in ˜30% of human breast and ovarian cancers, ErbB2 function is inhibited by the Herceptin® monoclonal antibody, alone and in combination with chemotherapeutics. The epidermal growth factor receptor (EGFR) is aberrantly expressed in e.g., colon cancers and head and neck cancers. EGFR is inhibited by the Erbitux® monoclonal antibody. Small molecule inhibitors of EGFR have also been approved (Tarceva® and Iressa®). Glivec® targets the Bcr-Abl fusion protein which causes chronic myelogenous leukemia (CML). Glivec® also targets the c-Kit receptor that is overexpressed in a rare form of gastric cancer (GIST). MUC1 is aberrantly overexpressed in more than 800,000 cancers each year in the United States.

B. Mucins

Mucin-type glycoproteins are normally expressed on the ductal cell surface of glandular epithelia. As major components of the mucosal surface that interfaces with the external environment, they constitute a protective barrier against damage induced by proteases, toxins, microorganisms and other forms of stress. The mucin-type glycoprotein family includes 20 known members (MUC1-MUC20) that are encoded by distinct genes. The common structural feature is variable numbers of tandem repeats with a high proportion of serines and threonines that are extensively modified by O-glycosylation. The secreted mucins MUC2, MUC5AC, MUC5B, MUC6) form the mucous gel that protects the epithelial cell surface. The membrane-bound mucins MUC1, MUC4, MUC13, MUC16) also contribute to formation of the mucous gel through their glycosylated ectodomains that extend from the apical cell surface. In addition, the membrane-bound mucins have hydrophobic transmembrane domains and cytoplasmic tails that can signal the presence of stress at the cell surface to the interior of the cell.

The Mucin 1 (MUC1) heterodimeric transmembrane glycoprotein is aberrantly overexpressed by MUC1 polypeptide is expressed as two subunits that form a stable heterodimer. The MUC1 N-terminal subunit (MUC1-N) consists in large part of variable numbers of 20 amino acid tandem repeats. MUC1-N is tethered to the cell surface by binding to the transmembrane MUC1 C-terminal subunit (MUC1-C). The physiologic function of MUC1 is to protect normal epithelial cells that are in contact with the external environment. MUC1-N extends beyond the cell glycocalyx as part of a physical barrier that protects epithelial cells from damage induced by toxins, infections, free radicals, acids and other forms of stress. MUC1-N is also shed into this protective barrier, leaving MUC1-C at the cell surface as a putative receptor for signaling the presence of stress to the interior of the cell. In turn, MUC1-C transduces signals to the inside of the cell that protect against death. Tumors have exploited, this function by overexpressing MUC1 to protect themselves against adverse growth conditions. Tumors that overexpress MUC1 are also resistant to treatment with anti-cancer agents. Importantly, overexpression of MUC1 is sufficient to cause malignant transformation.

MUC1 is overexpressed in human malignancies as a heterodimer with MUC1-N having the characteristic variable numbers of tandem repeats that arc extensively modified by O-linked glycans. The MUC1-C subunit was believed to function primarily in tethering MUC1-N at the cell membrane. However, the findings that MUC1-C interacts with diverse signaling molecules, localizes to the nucleus and mitochondria and induces transformation have generated interest, in the structure and function of this subunit. Similarly, by analogy to molecules such as ErbG2 and EGRF, the possibility of antagonizing interactions between the MUC1-N, or external domain, is intriguing.

### SUMMARY OF THE INVENTION

Thus, in accordance with the present invention, there is provided 1. A method of inhibiting a MUC1-positive cancer cell comprising contacting said cell with a MUC1 ligand trap, said ligand trap comprising (a) a first MUC1 segment comprising least a portion of a MUC1 external domain (ED); (b) at least a portion of an immunoglobulin Fc domain; and either or both of (c) as first linker disposed between (a) and (b) and/or glycosylation of residue Asn36 of the MUC1 ECD amino acid sequence. The first MUC1 segment may comprise at least 50 residues, may lacks tandem repeats, and/or may comprise the MUC1 SEA domain. The Fc domain portion may comprise a constant region from IgG1 or IgG2a, Contacting may be achieved by delivery of a viral expression vector encoding said ligand trap under the control of a promoter operable in said cancer cell.

The cancer cell may be a solid tumor cell, such as a lung cancer cell, a brain cancer cell, a head & neck cancer cell, a breast cancer cell, a skin cancer cell, a liver cancer cell, a pancreatic cancer cell, a stomach cancer cell, a colon cancer cell, a rectal cancer cell, a uterine cancer cell, a cervical cancer cell, an ovarian cancer cell, a testicular cancer cell, a skin cancer cell or an esophageal cancer cell. The cancer cell may be a leukemia or myeloma cell, such as an acute myeloid leukemia, chronic myelogenous leukemia or multiple myeloma.

The first linker may contain between 6 and 30 residues. The N- and C-termini of said first linker may be (I) L and AAA, respectively, on a flexible linker and (ii) LEA and AAA on a helical linker. The first linker may comprise the sequence GGGGSGGGGSGGGGS (SEQ ID NO: 10). The glycosylation may be an N-linked glycan comprising β-galactosidase.

The method may further comprise contacting said cancer cell with a second anti-cancer agent. The second anti-cancer agent may be contacted prior to or after said ligand trap, or at the same time as said ligand trap. The second anti-cancer agent may be selected from radiation, chemotherapy, immunotherapy, toxin therapy and hormonal therapy. Inhibiting may comprise inhibiting cancer coil growth or proliferation, or may comprise inducing cancer cell death, such as by apoptosis.

The ligand trap may comprise a second MUC1 segment comprising at least a portion of a MUC1 ED. The first MUC1 segment and said second MUC1 segment may be separated from each other by a second linker, such as GGGG (SEQ ID NO: 11). The ligand trap may comprise SEQ ID NO: 12, which is N-terminal to SEQ ID NO: 9, which is N-terminal to said portion of said immunoglobulin Fc domain, optionally where SEQ ID: NO 11 is disposed between SEQ ID NO: 12 and SEQ ID NO: 9, and SEQ ID NO: 10 is disposed between SEQ ID NO: 9 said immunoglobulin Fc domain. The ligand trap may comprise SEQ ID NO: 13 or 15.

In another embodiment, there is provided a method of inhibiting a MUC1-positive cancer in a subject comprising administering to said subject a MUC1 ligand trap, said ligand trap comprising (a) a first MUC1 segment comprising at least a portion of a MUC1 external domain (ED); (b) at least a portion of an immunoglobulin Fc domain; and either or both of (c) a first linker disposed between (a) and (b) and/or glycosylation of residue Asn36 of the MUC1 ECD amino acid sequence. The first MUC1 segment may comprise at least 50 residues, may lacks tandem repeats, and/or may comprise the MUC1 SEA domain. The Fc domain portion may comprise a constant region from IgG1 or IgG2a. Contacting may be achieved by delivery of a viral expression vector encoding said ligand trap under the control of a promoter operable in a cell of said subject.

The cancer may be a solid tumor, such as a lung cancer, a brain cancer, a head & neck cancer, a breast cancer, a skin cancer, a liver cancer, a pancreatic cancer, a stomach cancer, a colon cancer, a rectal cancer, a uterine cancer, a cervical cancer, an ovarian cancer, a testicular cancer, a skin cancer or an esophageal cancer. The cancer may be a leukemia or myeloma, such as an acute myeloid leukemia, chronic myelogenous leukemia or multiple myeloma.

The first linker may contain between 5 and 30 residues. The N- and C-termini of said first linker may be (i) L and AAA, respectively, on a flexible linker and (ii) LEA and AAA on a helical linker. The first linker may comprise the sequence GGGGSGGGGSGGGGS (SEQ ID NO:10). The glycosylation may be an N-linked glycan comprising β-galactosidase.

The method may further comprising providing said subject with a second anti-cancer therapy. The second anti-cancer therapy may be provided prior to said ligand trap or after said ligand trap, or at the same time as said ligand trap. The second anti-cancer Therapy is selected from surgery, radiation, chemotherapy, immunotherapy, toxin therapy and hormonal therapy. Inhibiting may comprise inhibiting cancer cell growth or proliferation, or may comprise inducing cancer cell death, such as by apoptosis. Administering may comprise intravenous, intra-arterial, oral, intratumoral, subcutaneous, topical or intraperitoneal administration, or may comprise local, regional, systemic, or continual administration.

The ligand trap may comprise a second MUC1 segment comprising at least a portion of a META ED. The first MUC1 segment and said second MUC1 segment may be separated from each other by a second linker, such as GGGG (SEQ ID NO: 11). The ligand trap may comprise SEQ ID NO: 12, which is N-terminal to SEQ ID NO: 9, which is N-terminal to said portion of said immunoglobulin Fc domain, optionally where SEQ ID: NO 11 is disposed between SEQ ID NO: 12 and SEQ ID NO: 9, and SEQ ID NO: 10 is disposed between SEQ ID NO: 9 said immunoglobulin Fc domain. The ligand trap may comprise SEQ ID NO: 13 or 15.

In yet another embodiment, there is provided a pharmaceutical composition comprising a MUC1 ligand trap, said ligand trap comprising (a) a first MUC1 segment comprising at least a portion of a MUC1 external domain (ED); (b) at least a portion of an immunoglobulin Fc domain; and either or both of (c) a first linker disposed between (a) and (h) and/or glycosylation of residue Asn36 of the MUC1 ECD amino acid sequence. The first MUC1 segment comprises at least 15 residues, may lack tandem repeats, and/or may comprise the MUC1 SEA domain. The Fc domain portion may comprise an IgG1 or IgG2a constant region. The first linker may contain between 6 and 30 residues. The first linker may comprise the sequence GGGGSGGGGSGGGGS (SEQ ID NO:10) and/or said glycosylation is an N-linked glycan comprising beta-galactosides. The ligand trap may be encapsulated or embedded in a delivery vehicle, such as a liposome, a lysosome, a microcapsule or a nanoparticle. The N- and C-termini of the linker may be (i) L and AAA, respectively, on a flexible linker and (ii) LEA and AAA on a helical linker. The ligand trap may have the sequence of SEQ ID NO:1 or SEQ ID NO:2.

The ligand trap may comprise a second MUC1 segment comprising at least a portion of a MIXT ED. The first MUC1 segment and said second MUC1 segment may be separated from each other by a second linker, such as GGGG (SEQ ID NO: 11). The ligand trap may comprise SEQ ID NO: 12, which is N-terminal to SEQ ID NO: 9, which is N-terminal to said portion of said immunoglobulin Fc domain, optionally where SEQ ID: NO 11 is disposed between SEQ ID NO: 12 and SEQ ID NO: 9, and SEQ ID NO: it) is disposed between SEQ ID NO: 9 said immunoglobulin Fc domain. The ligand trap may comprise SEQ ID NO: 13 or 15.

It is contemplated that any method or composition described herein can be implemented with respect to any other method or composition described herein.

The use of the word “a” or “an” when used in conjunction with the term “comprising” in the claims and/or the specification may mean “one” but it is also consistent with the meaning of “one or more,” “at least one,” and “one or more than one.” The word “about” means plus or minus 5% of the stated number.

Other objects, features and advantages of the present invention will become apparent from the following detailed description. It should be understood, however, that the detailed description and the specific examples, while indicating specific embodiments of the invention, are given by way of illustration only since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed.

FIG. 1. Schematic representation of the transmembrane MUC1-C subunit and amino acid sequence (SEQ ID NO:9) of the extracellular domain (MUC1-ED).

FIG. 2. GO-101 (hFc-MUC1-C-ED) purified proteins from different batches 4: generated at Genus Oncology; 2: produced at Xtal BioStructures Inc., and 3: produced at HyproCell Inc.,) were resolved by SDS-PAGE and stained with coomassie blue staining.

FIG. 3. Construction of MUC1-COED-linker-hFc construct. The cDNA fragment containing signal sequence along with p58 MUC1-C-ED fragment, Gly/Ser linker sequences were obtained by PCR amplification using a previous version of the construct containing mIg-Fc fusion as template. Human Ig-Fc fragment was amplified from the Invitrogen vector (pFUSE-hIgG2-Fc2). Both of these fragments were fused together using overlapping primers for both the fragments and amplified as one piece which contains signal sequence, p58 MUC1-C-ED, Gly/Ser linker and hIg-Fc fragments in tandem. Following appropriate restriction digestion, the insert was ligated back to the pCR3.1 vector.

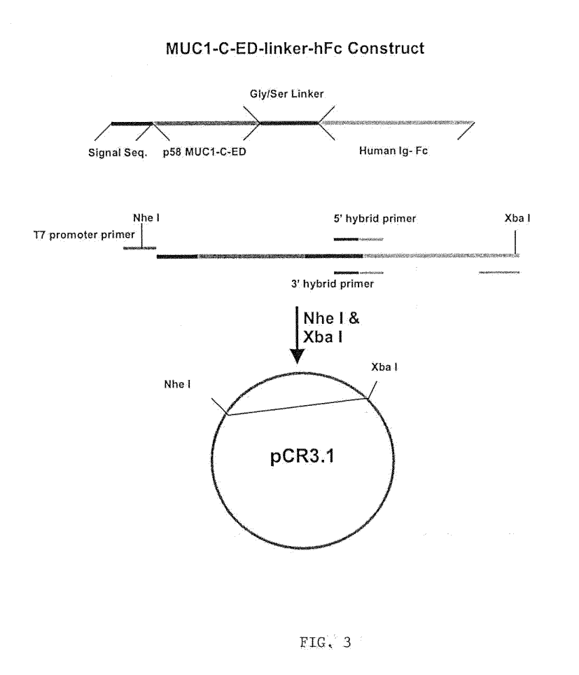

FIG. 4. ZR-75-1 cells were treated with the indicated concentrations of GO-101 each day for 3 days. The cell proliferation was measured by trypan blue exclusion method. The percent proliferation is shown to that compared with control.

FIG. 5. MCF-7 cells were treated with the indicated concentrations of GO-101 each day for 3 days. The cell proliferation was measured by trypan blue exclusion method. The percent proliferation is shown to that normalized with control.

FIG. 6. H-1975 non-small cell lung carcinoma cells were treated with the indicated concentrations of GO-101 each day for 3 days. The cell proliferation was measured by trypan blue exclusion method. The percent proliferation is shown to that compared with control.

FIG. 7. H1650 cells in a 96-well plate were treated with various concentrations of purified of GO-101 for four days. AlamarBlue dye was added to the cells on day 5, and the absorbance was measured at 570 and 600 nm. A 4-parameter curve was obtained by plotting the percentage reduction of AlamarBlue calculated using these absorbance values against the concentrations of GO-101.

FIG. 8. ZR-75-1 human breast carcinoma cells were treated with 500 nM GO-101 and/or 1 mM H₂O₂ for 3 days and the cell proliferation was measured by trypan blue exclusion method. The percent proliferation is shown to that compared with control.

FIG. 9. ZR-75-1 breast carcinoma cells in a 96-well plate were treated with various concentrations of purified of GO-101 (p59, red curve), multiple concentrations of Doxorubicin (blue curve) and combinations of GO-101 and Doxorubicin (green curve) for four days. AlamarBlue dye was added to the cells on day 5, and the absorbance was measured at 570 and 600 nm. A 4-parameter curve was obtained by plotting the percentage reduction of alamarBlue calculated using these absorbance values against respective treatment concentrations.

FIG. 10. ZR-75-1 human breast carcinoma bearing nu/nu mice were dosed with either vehicle (squares), 1 mg/kg, GO-101 i.p daily×21 days (triangles) or 10 mg/kg GO-101 i.p. twice weekly for 3 weeks. Tumors were measured twice a week.

FIG. 11. ZR-75-1 human breast carcinoma bearing nu/nu mice were dosed with either doxorubicin alone (squares) or in combination with 1 mg/kg GO-101 i.p daily×21 days (circles). Tumors were measured twice a week.

FIG. 12. ZR-75-1 Hormone-dependent Breast Carcinoma Xenograft Model: Preliminary 2nd TRAP Study (mean tumor volume+SE). Five- to 6-wk-old female BALB/c nu/nu mice were implanted with 17β-estradiol plugs. After 24 hrs, ZR-75-1 human breast carcinoma cells (imbedded in Matrigel) were injected s.c. in the flank. When tumors were ˜100 mm³, the mice were pair matched into groups of 4 mice and injected i.p. with PBS (black squares) or with 10 mg/kg MUC1-Link-Trap (Red squares) each day for 21 days. Mice were weighed 2-3 times a week and tumor measurements were also performed 2-3 times a week. Points, mean tumor volumes±SE.

FIG. 13. ZR-75-1 Hormone-dependent Breast Carcinoma Xenograft Model: Preliminary 2nd TRAP Study (individual mice tumor volume). Five- to 6-wk-old female BALB/c nu/nu mice were implanted with 17β-estradiol plugs. After 24 hrs, ZR-75-1 human breast carcinoma cells (imbedded in Matrigel) were injected s.c. in the flank. When tumors were ˜100 mm³ (individual range of 64-185 mm³), the mice were pair matched into groups of 4 mice and injected i.p. with PBS (black squares) or with 10 mg/kg MUC1-Link-Trap (Red squares) each day for 21 days. Mice were weighed 2-3 times a week and tumor measurements were also performed 2-3 times a week. (left panel): Tumor volumes from vehicle-treated individual mice; (right panel): Tumor volumes from MUC1-Link-Trap-treated individual mice.

FIG. 14. ZR-75-1 Hormone-dependent Breast Carcinoma Xenogaft Model: Preliminary 2nd TRAP Study (median tumor volume±SE). Five- to 6-wk-old female BALB/c nu/nu mice were implanted with 17β-estradiol plugs. After 24 hrs, ZR-75-1 human breast carcinoma cells (imbedded in Matrigel) were injected s.c. in the flank. When tumors were ˜100 mm³, the mice were pair matched into groups of 4 mice and injected i.p. with PBS (black squares) or with 10 mg/kg MUC1-Link-Trap (Red squares) each day for 21 days. Mice were weighed 2-3 times a week and tumor measurements were also performed 2-3 times a week. Points, median tumor volumes are shown in the graph.

FIG. 15. ZR-75-1 Hormone-dependent Breast Carcinoma Xenograft Model: Preliminary 2nd TRAP Study (mice body weights). Five- to 6-wk-old female BALB/c nu/nu mice were implanted with 17β-estradiol plugs. After 24 hours, ZR-75-1 human breast carcinoma cells (imbedded in Matrigel) were injected s.c. in the flank. When tumors were ˜100 mm³, the mice were pair matched into groups of 4 mice and injected i.p. with PBS (black squares) or with 10 mg/kg MUC1-Link-Trap (Red squares) each day for 21 days. Mice were weighed 2-3 times a week. Points, mean body weights of mice are shown in the graph.

FIG. 16. ZR-75-1 Hormone-dependent Breast Carcinoma Xenograft Model: Preliminary 1st TRAP Study (mean tumor volume+SE). ZR-75-1 human breast carcinoma hearing nu/nu mice were dosed with either vehicle (squares), 1 mg/kg GO-101 i.p daily×21 days (triangles) or 10 mg/kg GO-101 i.p. twice weekly (circles) for 3 weeks, Mice were weighed 2-3 times a week and tumor measurements were also performed 2-3 times a week. Points, mean tumor volumes±SE.

FIG. 17. ZR-75-1 Hormone-dependent Breast Carcinoma Xenograft Model: Preliminary 1st TRAP Study in Combination with Doxorubicin (mean tumor volume+SE). ZR-75-1 human breast carcinoma bearing nu/nu mice were dosed with either doxorubicin 6 mg/kg i.v. Q4D×3 (squares) or 1 mg/kg GO-101 i.p daily×21 days+doxorubicin 6 mg/kg i.v. Q4D×3 (circles). Mice were weighed 2-3 times a week and tumor measurements were also performed 2-3 times a week. Points, mean tumor volumes±SE.

FIG. 18. Coimmunoprecipitation of cell lysates expressing MUC1-C. MUC1-C was transiently expressed in wild-type CHO-K1 cells or the glycosylation-deficient Lec1 and Lec8 variants, Lysates were immunoblotted with anti-MUC1-C (left). The lysates were also incubated with GST-galectin-3 and the precipitates immunoblotted with anti-MUC1-C (right).

FIG. 19. Modified MUC1-TRAP protein with the SEA domain (62 amino acid). From left to right, the top construct includes MUC-1 signal sequence linked to the 62 amino acid MUC1-SEA domain, then the short 4G linker, then the MUC1-ECD (58 residues) domain, followed by the G/S linker and then the mouse immunoglobulin Fc domain. The botton construct differs only in the that the IL-2 signal sequence is used.

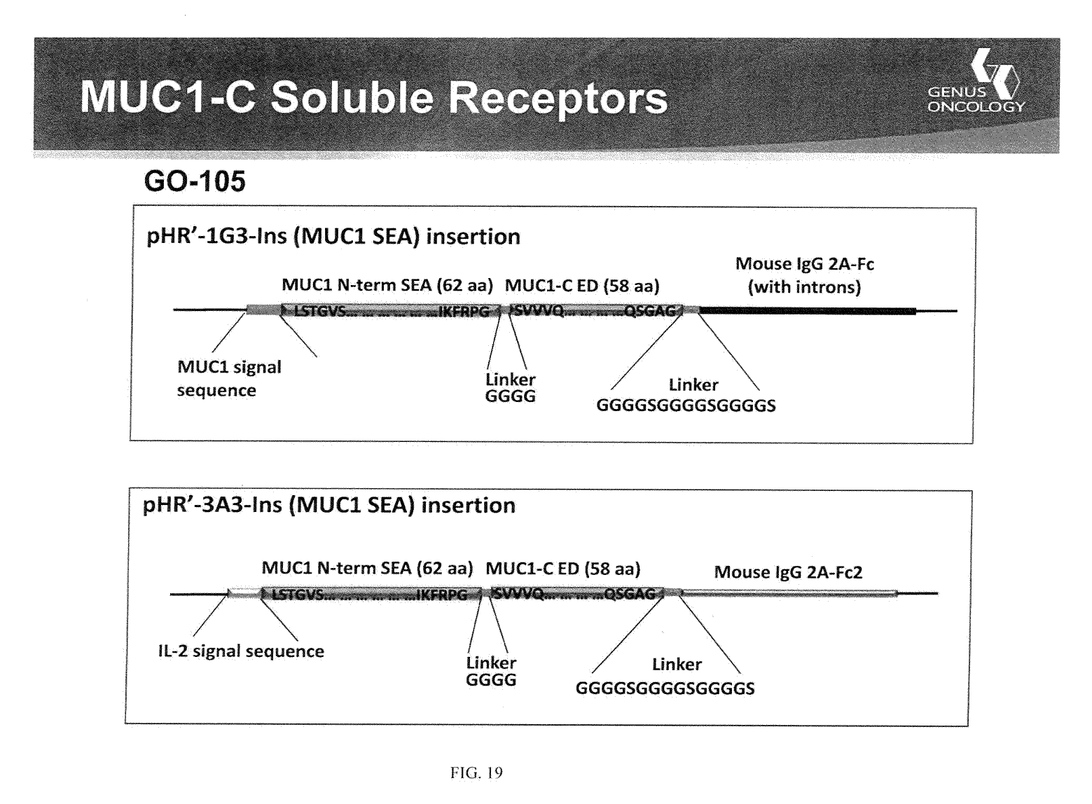

### DESCRIPTION OF ILLUSTRATIVE EMBODIMENTS

MUC1 has been studied extensively by the inventors and others for its role in cancer. Human MUC1 is heterodimeric glycoprotein, translated as a single polypeptide and cleaved into N- and C-terminal subunits in the endoplasmic reticulum (Ligtenberg et al., 1992; Macao et al., 2006; Levitin et al., 2005), Aberrant overexpression of MUC1, as found in most human carcinomas (Kufe et al., 1984), confers anchorage-independent growth and tumorigenicity (Li et al., 2003a; Huang et al., 2003; Schroeder et al., 2004; Huang et al., 2005). Other studies have demonstrated that overexpression of MUC1 confers resistance to apoptosis induced by oxidative stress and genotoxic anti-cancer agents (Yin and Kufe, 2003; Ren et al., 2004; Raina et al., 2004; Yin et al., 2004; Raina et al., 2006; Yin et al., 2007).

Tethered and secreted mucins function in providing a protective barrier of the epithelial cell surface. With damage to the epithelial layer, the tight junctions between neighboring cells are disrupted, and polarity is lost as the cells initiate a heregulin-induced repair program (Vermeer et al., 2003.) MUC1-N is shed from the cell surface (Abe and Kufe, 1989), leaving MUC1-C to function as a transducer of environmental stress signals to the interior of the cell. In this regard. MUC1-C forms cell surface complexes with members of the ErbB receptor family, and MUC1-C is targeted to the nucleus in the response to heregulin stimulation (Li et al., 2001; Li et al., 2003c). MUC1-C also functions in integrating the ErbB receptor and Wnt signaling pathways through direct interactions between the MUC1 cytoplasmic domain (CD) and members of the catenin family (Huang et al., 2005; Li et al., 2003c; Yamamoto et al., 1997; Li et al., 1998; Li et al., 2001; Li and Kufe, 2001). Other studies have demonstrated that MUC1-CD is phosphorylated by glycogen synthase kinase 3β, c-Src, protein kinase Cδ, and c-Abl (Raina et al., 2006; Li et al., 1998; Li et al., 2001; Ren et al., 2002).

In 2006, the inventors reported that MUC1 is imported into the nucleus by a mechanism involving binding to Nup62 (Leng et al., 2007). They also demonstrate that MUC1 forms oligomers through a CQC motif in the MUC1 cytoplasmic domain and that MUC1 oligomerization is necessary for nuclear import. In 2007, they also demonstrated that overexpression of MUC1 in human carcinoma cells is associated with constitutive activation of NF-kappaB p65 (Ahmad et al., 2007.) MUC1 was shown to interact with the high-molecular-weight IκB kinase (IKK) complex in vivo, and that the MUC1 cytoplasmic domain binds directly to IKKβ and IKKγ. Interaction of MUC1 with both IKKβ and IKKγ is necessary for IKKβ activation, resulting in phosphorylation and degradation of IκBα. These findings indicated that MUC1 is important for physiological activation of IKKβ and that overexpression of MUC1, as found in human cancers, confers sustained induction of the IKKβ-NF-κB p65 pathway.

The inventors have also examined the role of MUC1 in inflammatory disease states. Studies demonstrate that MUC1-CD binds directly to NF-κB p65 and blocks the interaction between NF-κB p65 and IκBα. The inventors also showed that the MUC1-C subunit associates with NF-κB p65 on the promoters of NF-κB target genes and promotes NF-κB-mediated transcription. They also demonstrated that an inhibitor of MUC1-C oligomerization blocks the MUC1 interaction with NF-κB p65 and constitutive activation of the inflammatory NF-κB pathway in addition, a similar interaction with STAT3, another inflammatory signaling factor, was demonstrated, even further implicating MUC1 in this process.

In the work described below, the inventors now expand their studies of the external domain (ED) of MUC1 as a therapeutic target. In earlier work, the inventors described a simple ligand trap which employed a direct fusion of a portion of MUC1 ED to immunoglobulin Fc (see U.S. Pat. No. 8,129,506). Unfortunately, the activity of this construct was less than hoped for. Now, the inventors have developed an improved ligand trap made up of a portion of the MUC1 ED and an Ig Fc domain, this time connected by a flexible linker molecule. As illustrated below, this construct shows remarkable anti-cancer activity both in vitro and in vivo well beyond the first generation technology described above. These and other aspects of the invention are described in further detail, below.

### I. MUC1

A. Structure

MUC1 is a 158 amino acid mucin-type glycoprotein with a predicted molecular mass of 17 kDa that is expressed on the apical borders of normal secretory epithelial cells (Kufe et al., 1984). In this regard, MUC1-C is also detectable as a 17 kDa species, the electrophoretic mobility of which is not affected by N-glycosidases or expression in glycosylation-deficient cells. MUC1 forms a heterodimer following synthesis as a single polypeptide and cleavage of the precursor into two subunits in the endoplasmic reticulum (Ligtenberg et al., 1992). The cleavage may be mediated by an autocatalytic process (Levitan et al., 2005). The >250 kDa MUC1 N-terminal (MUC1 N-ter, MUC1-N) subunit contains variable numbers of 20 amino acid tandem repeats that are imperfect with highly conserved variations and are modified by O-linked glycans (Gendler et al., 1988; Siddiqui et al., 1988), MUC1-N is tethered to the cell surface by dimerization with the ˜23 kDa C-terminal subunit (MUC1 C-ter, MUC1-C), which includes a 59 amino acid extracellular region, a 28 amino acid transmembrane domain and a 72 amino acid cytoplasmic domain (CD) (Menlo et al., 1989). The human MUC1 sequence is shown below:

(SEQ ID NO: 2)
GSVVVQLTLAFREGTINVHDVETQFNQYKTEAASRYNLTISDVSVSDVP
FPFSAQSGAGVPGWGIALLVLVCVLVALAIVYLIALAVCQCRRKNYGQL
DIFPARDTYHPMSEYPTYHTHGRYVPPSSTDRSPYEKVSAGNGGSSLSY
TNPAVAATSANL

The double-underlined sequence is the 59 residue ED. The bold sequence indicates the CD. With transformation of normal epithelia to carcinomas, MUC1 is aberrantly overexpressed in the cytosol and over the entire cell membrane (Kufe et al., 1984: Percy et al., 1992). Cell membrane-associated MUC1 is targeted to endosomes by clathrin-mediated endocytosis (Kinlough et al., 2004). In addition, MUC1-C, but not MUC1-N, is targeted to the nucleus (Baldus et al., 2004; Huang et al., 2003; Li et al., 2003a; Li et al., 2003b; Li et al., 2003c; Wei et al., 2005; Wen et al., 2003) and mitochondria (Ren et al., 2004).

Studies by the inventors have demonstrated that MUC1-C is endogeneously expressed as a 20-25 kDa form that is glycosylated on Asn₃₆, Treatment of MUC1-C with N-glycosidase, expression of MUC1 in N-glycosylation-deficient CHO cells and mutation of MUC1-C at Asn₃₆ were each associated with loss of the 20-25 kDa species. Consistent with the MUC1-C 20-25 kDa form being the glycosylated product of the 17 kDa backbone, digestion with N-glycosidases was associated with conversion of the 20-25 kDa glycoprotein to the 17 kDa protein.

B. Function

MUC1 interacts with members of the ErbB receptor family (Li et al., 2001b; Li et al., 2003c; Schroeder et al., 2001) and with the Wnt effector, β-catenin (Yamamoto et al., 1997). The epidermal growth factor receptor and c-Src phosphorylate the MUC1 cytoplasmic domain (MUC1-CD) on Y-46 and thereby increase binding of MUC1 and β-catenin (Li et al., 2001a, Li et al., 2001b). Binding of MUC1 and β-catenin is also regulated by glycogen synthase kinase 3β and protein kinase Cδ (Li et al., 1998; Ren et al., 2002). MUC1 colocalizes with β-catenin in the nucleus (Baldus et al., 2004; Li et al., 2003a; Li et al., 2003c; Wen et al., 2003) and coactivates transcription of Wnt target genes (Huang et al., 2003). Other studies have shown that MUC1 also binds directly to p53 and regulates transcription of p53 target genes (Wei et al., 2005). Notably, overexpression of MUC1 is sufficient to induce anchorage-independent growth and tumorigenicity (Huang et al., 2003; Li et al., 2003b; Ren et al., 2002; Schroeder et al., 2004).

Most mitochondrial proteins are encoded in the nucleus and are imported into mitochondria by translocation complexes in the outer and inner mitochondrial membranes. Certain mitochondrial proteins contain N-terminal mitochondrial targeting sequences and interact with Tom20 in the outer mitochondrial membrane (Truscott et al., 2003). Other mitochondrial proteins contain internal targeting sequences and interact with the Tom70 receptor (Truscott et al., 2003). Recent work showed that mitochondrial proteins without internal targeting sequences are delivered to Tom70 by a complex of HSP70 and HSP90 (Young et al., 2003).

### II. MUC1 LIGAND TRAPS

A. Structure

The present invention contemplates the design, production and use of various MUC1 ligand traps. The contemplated ligand traps will have three elements: at least a portion of the MUC1-ED, a linkers, and at least a portion of an immunoglobulin Fc sequence. Each of these elements is described in greater detail below.

In general, the peptides will be 50 residues or more in length comprising consecutive residues of MUC1-ED. The overall length may be 50, 60, 70, 80, 90, 100 or more residues. Ranges of peptide length of 50-60 residues, 50-70 residues, 50-80 residues 50-90, residues, 50-100 residues, 50-75 residues and 75-100 residues are contemplated. The number of consecutive MUC1 residues may be 10, 11, 12, 13, 14, 15, 16, 17, 18, 19, 20, 25, 10, 35, 40, 45, 50, 51, 52, 53, 54, 55, 56, 57, 58 or 59 residues. Ranges of consecutive residues of 10-20 residues, 15-20 residues, 15-25 residues, 10-30 residues, 10-40 residues, 10-50 residues, 10-59 residues and 20-59 residues are contemplated.

The present invention may utilize L-configuration amino acids, D-configuration amino acids, or a mixture thereof. While L-amino acids represent the vast majority of amino acids found in proteins. D-amino acids are found in some proteins produced by exotic sea-dwelling organisms, such as cone snails. They are also abundant components of the peptidoglycan cell walls of bacteria. D-serine may act as a neurotransmitter in the brain. The L and D convention for amino acid configuration refers not to the optical activity of the amino acid itself, but rather to the optical activity of the isomer of glyceraldehyde from which that amino acid can theoretically be synthesized (D-glyceraldehyde is dextrorotary; L-glyceraldehyde is levorotary).

One form of an “all-D” peptide is a retro-inverso peptide. Retro-inverso modification of naturally occurring polypeptides involves the synthetic assemblage of amino acids with α-carbon stereochemistry opposite to that of the corresponding L-amino acids, i.e., D-amino acids in reverse order with respect to the native peptide sequence. A retro-inverso analogue thus has reversed termini and reversed direction of peptide bonds (NH—CO rather than CO—NH) while approximately maintaining the topology of the side chains as in the native peptide sequence. See U.S. Pat. No. 6,261,569, incorporated herein by reference.

As mentioned above, peptides modified for in vivo use by the addition, at the amino- and/or carboxyl-terminal ends, of a blocking agent to facilitate survival of the peptide in vivo arc contemplated. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. These agents can be added either chemically during the synthesis of the peptide, or by recombinant DNA technology by methods familiar in the art. Alternatively, blocking agents such as pyroglutamic acid or other molecules known in the art can be attached to the amino and/or carboxyl terminal residues.

The Fc (fragment, crystallizable) region of immunoglobulin interacts with the Fc receptor on certain cells. The constant region is identical in all antibodies of the same isotype, but differs in antibodies of different isotypes. There are five types of mammalian Ig heavy chains, denoted by the Greek letters: α, δ, ε, γ, and μ, the constant regions of which dictate the structure of the Fc. Distinct heavy chains differ in size and composition; α and γ contain approximately 450 amino acids, while μ and ε have approximately 550 amino acids. Heavy chains γ, α and δ have a constant region composed of three tandem (in a line) Ig domains, and a hinge region for added flexibility; heavy chains μ and ε have a constant region composed of four immunoglobulin domains.

The MUC1-ED-Trap can be the 1-59 amino acids of MUC1-ED N-terminal to the plasma membrane domain fused with the constant region (Fc) of human or mouse IgG1. Additional MUC1-ED-Traps can be created where the constant region (Fc) of human or mouse IgG1 are fused with different portions of MUC1-ED (1-59 aa) and spaced with a linker sequences. Several other MUC1-ED-Traps can be used in which the highly positively charged amino acids from the MUC1-ED 1-59 domain can be excised. Moreover, a minor stretch of highly basic amino acids in MUC1-ED 1-59 can be deleted to generate a variant MUC1-ED-Trap for better PK characteristics.

Linkers or cross-linking agents may be used to fuse MUC1-ED segments to the constant region (Fc) of human or mouse IgG1 sequences, Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved, to be highly efficient in inducing cross-linking between identical and different macromolecules or subunits of a macromolecule, and linking of polypeptide ligands to their specific binding sites. Heterobifunctional reagents contain two different functional, groups. By taking advantage of the differential reactivities of the two different functional groups, cross-linking can be controlled both selectively and sequentially. The bifunctional cross-linking reagents can be divided according to the specificity of their functional groups, e.g., amino-, sulfhydryl-, guanidino-, indole-, or carboxyl-specific groups. Of these, reagents directed to free amino groups have become especially popular because of their commercial availability, case of synthesis and the mild reaction conditions under which they can be applied. A majority of heterobifunctional cross-linking reagents contains a primary amine-reactive group and a thiol-reactive group.

In another example, heterobifunctional cross-linking reagents and methods of using the cross-linking reagents are described in U.S. Pat. No. 5,889,155, specifically incorporated herein by reference in its entirety. The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides. In instances where a particular peptide does not contain a residue amenable for a given cross-linking reagent in its native sequence, conservative genetic or synthetic amino acid changes in the primary sequence can be utilized.

The inventors constructed the Fc-MUC1-p59 chimeric protein connected by a (GGGGS)₃ linker. This chimeric protein can also be constructed by flexible linkers such as (GGGGS)ₙ where n=2-5. Moreover, helical linkers such as (EAAAK)ₙ where n=2-6 can also be used to provide proper conformation to the chimeric, protein. The various sequences of the flexible linkers can be:

(SEQ ID NO: 3)
GGGGS GGGGS
(SEQ ID NO: 10)
GGGGS GGGGS GGGGS
(SEQ ID NO: 4)
GGGGS GGGGS GGGGS GGGGS
(SEQ ID NO: 5)
GGGGS GGGGS GGGGS GGGGS GGGGS

The various sequences of the helical linkers can be:

(SEQ ID NO: 6)
EAAAK EAAAK
(SEQ ID NO: 7)
EAAAK EAAAK EAAAK
(SEQ ID NO: 8)
EAAAK EAAAK EAAAK EAAAK

Other combinations are contemplated as well.

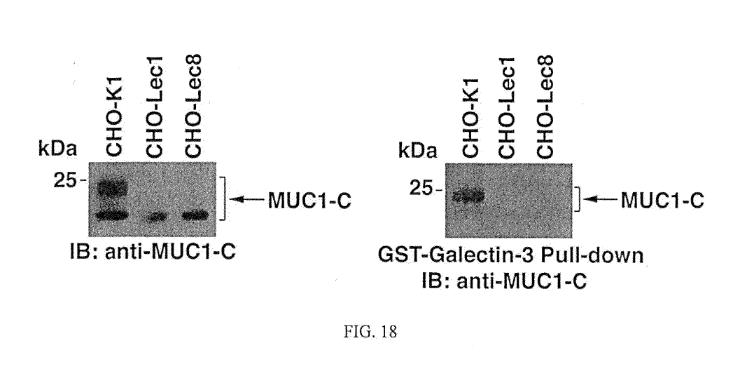

Alternatively or in addition to the linker described above, the ligand trap may include a glycosylation modification, in particular at what corresponds to residue Asn36 of the MUC1 58 reside ECD sequence. In the native molecule, this structural feature has been shown to be important in binding of MUC1 to molecules such as galactin-3. EGFR and ErbB2. This requirement with respect to galectin-3 has been demonstrated for the ligand trap as well (see FIG. 18).

B. Chemical Synthesis

It will be may be advantageous to produce peptides using the solid-phase synthetic techniques (Merrifield, 1963). Other peptide synthesis techniques are well known to those of skill in the art (Bodanszky et al., 1976; Peptide Synthesis, 1985; Solid Phase Peptide Synthelia, 1984). Appropriate protective groups for use in such syntheses will be found in the above texts, as well as in Protective Groups in Organic Chemistry, 1973. These synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine.

Using solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence any remaining terminal and side group protecting groups (and solid support) are removed sequentially or concurrently, to provide the final peptide. The peptides of the invention are preferably devoid of benzylated or methylbenzylated amino acids. Such protecting group moieties may be used in the course of synthesis, but they are removed before the peptides are used. Additional reactions may be necessary, as described elsewhere, to form intramolecular linkages to restrain conformation.

Aside from the twenty standard amino acids can be used, there are a vast number of “non-standard” amino acids. Two of these can be specified by the genetic code, but are rather rare in proteins. Selenocysteine is incorporated into some proteins at a UGA codon, which is normally a stop codon. Pyrrolysine is used by some methanogenic archaea in enzymes that they use to produce methane. It is coded for with the codon UAG. Examples of non-standard amino acids that are not found in proteins include lanthionine, 2-aminoisobutyric acid, dehydroalanine and the neurotransmitter gamma-aminobutyric acid. Non-standard amino acids often occur as intermediates in the metabolic pathways for standard amino acids—for example ornithine and citrulline occur in the urea cycle, part of amino acid catabolism. Non-standard amino acids arc usually formed through modifications to standard amino acids. For example, homocysteine is formed through the transsulfuration pathway or by the demethylation of methionine via the intermediate metabolite S-adenosyl methionine, while hydroxyproline is made by a posttranslational modification of praline.

C. Recombinant Production

In addition to chemical synthesis, the MUC1 ligand trap molecules of the present invention may advantageously be produced by recombinant methods. Nucleic acids according to the present invention will encode the MUC1 ligand trap, and optionally further include sequences. As used in this application, the term “a nucleic acid encoding a MUC1 ligand trap” refers to a nucleic; acid molecule that has been isolated free of total cellular nucleic acid. In certain embodiments, the invention concerns a nucleic acid encoding SEQ ID NO:1.

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The DNA segments of the present invention include those encoding biologically functional equivalent proteins and peptides of the sequences described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

Within certain embodiments, expression vectors are employed to express a MUC1 ligand trap in order to produce and isolate the polypeptide expressed therefrom. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation, of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous.” Similarly, an enhancer may be one naturally associated with as nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-Occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Table 2 lists several elements/promoters that may be employed, in the context of the present invention, to regulate the expression of a gene. This list is not intended to be exhaustive of all the possible elements involved in the promotion of expression but, merely, to be exemplary thereof. Table 3 provides examples of inducible elements, which are regions of a nucleic, acid sequence that can be activated in response to a specific stimulus.

TABLE 2
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton et |
| | al; 1990 |
| Immunoglobulin Light Chain | Queen et al., 1983; Picard et al., 1984 |
| T-Cell Receptor | Luria et al., 1987; Winoto et al., 1989; Redondo et al; |
| | 1990 |
| HLA DQ a and/or DQ β | Sullivan et al., 1987 |
| β-Interferon | Goodbourn et al., 1986; Fujita et al., 1987; Goodbourn et |
| | al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule (NCAM) | Hirsh et al., 1990 |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrook et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek et |
| | al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake et |
| | al., 1988; Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Choi et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987; Glue et al., 1988 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits et |
| | al., 1988; Feng et al., 1988; Takebe et al., 1988; Rosen et |
| | al., 1988; Berkhout et al., 1989; Laspia et al., 1989; Sharp |
| | et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa |
| | | et al., 1987, Karin et al., 1987; |
| | | Angel et al., 1987b; McNeall |
| | | et al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai |
| | | et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 Large T | Taylor et al., 1989, 1990a, 1990b |
| | Antigen | |
| Proliferin | Phorbol Ester-TPA | Mordacq et al., 1989 |
| Tumor Necrosis Factor | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Examples of such regions include the human LIMK2 gene (Nomoto et al., 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998). D1A dopamine receptor gene (Lee, et al., 1997), insulin-like growth factor II (Wu et al., 1997), human platelet endothelial cell adhesion molecule-1 (Almendro et al., 1996). Tumor specific promoters also will find use in the present invention. Some such promoters are set forth in Table 4.

TABLE 4
Candidate Tissue-Specific Promoters for Cancer Gene Therapy

| | Cancers in which promoter | Normal cells in which |
|---|---|---|
| Tissue-specific promoter | is active | promoter is active |
| Carcinoembryonic antigen | Most colorectal carcinomas; | Colonic mucosa; gastric |
| (CEA)* | 50% of lung carcinomas; | mucosa; lung epithelia; |
| | 40-50% of gastric carcinomas; | eccrine sweat glands; |
| | most pancreatic carcinomas; | cells in testes |
| | many breast carcinomas | |
| Prostate-specific antigen | Most prostate carcinomas | Prostate epithelium |
| (PSA) | | |
| Vasoactive intestinal peptide | Majority of non-small cell | Neurons; lymphocytes; mast |
| (VIP) | lung cancers | cells; eosinophils |
| Surfactant protein A (SP-A) | Many lung adenocarcinomas | Type II pneumocytes; Clara |
| | cells | |
| Human achaete-scute | Most small cell lung cancers | Neuroendocrine cells in lung |
| homolog (hASH) | | |
| Mucin-1 (MUC1)** | Most adenocarcinomas | Glandular epithelial cells in |
| | (originating from any tissue) | breast and in respiratory, |
| | | gastrointestinal, and |
| | | genitourinary tracts |
| Alpha-fetoprotein | Most hepatocellular | Hepatocytes (under certain |
| | carcinomas; possibly many | conditions); testis |
| | testicular cancers | |
| Albumin | Most hepatocellular | Hepatocytes |
| | carcinomas | |
| Tyrosinase | Most melanomas | Melanocytes; astrocytes; |
| | | Schwann cells; some neurons |
| Tyrosine-binding protein | Most melanomas | Melanocytes; astrocytes, |
| (TRP) | | Schwann cells; some neurons |
| Keratin 14 | Presumably many squamous | Keratinocytes |
| | cell carcinomas (e.g., Head | |
| | and neck cancers) | |
| EBV LD-2 | Many squamous cell | Keratinocytes of upper |
| | carcinomas of head and neck | digestive |
| | | Keratinocytes of upper |
| | | digestive tract |
| Glial fibrillary acidic protein | Many astrocytomas | Astrocytes |
| (GFAP) | | |
| Myelin basic protein (MBP) | Many gliomas | Oligodendrocytes |
| Testis-specific angiotensin- | Possibly many testicular | Spermatazoa |
| converting enzyme (Testis- | cancers | |
| specific ACE) | | |
| Osteocalcin | Possibly many osteosarcomas | Osteoblasts |
| E2F-regulated promoter | Almost all cancers | Proliferating cells |
| HLA-G | Many colorectal carcinomas; | Lymphocytes; monocytes; |
| | many melanomas; possibly | spermatocytes; trophoblast |
| | many other cancers | |
| FasL | Most melanomas; many | Activated leukocytes; |
| | pancreatic carcinomas; most | neurons; endothelial cells; |
| | astrocytomas possibly many | keratinocytes; cells in |
| | other cancers | immunoprivileged tissues; |
| | | some cells in lungs, ovaries, |
| | | liver, and prostate |
| Myc-regulated promoter | Most lung carcinomas (both | Proliferating cells (only some |
| | small cell and non-small cell); | cell-types): mammary |
| | most colorectal carcinomas | epithelial cells (including non- |
| | | proliferating) |
| MAGE-1 | Many melanomas; some non- | Testis |
| | small cell lung carcinomas; | |
| | some breast carcinomas | |
| VEGF | 70% of all cancers (constitutive | Cells at sites of |
| | overexpression in many cancers) | neovascularization (but unlike |
| | | in tumors, expression is |
| | | transient, less strong, and |
| | | never constitutive) |
| bFGF | Presumably many different | Cells at sites of ischemia (but |
| | cancers, since bFGF | unlike tumors, expression is |
| | expression is induced by | transient, less strong, and |
| | ischemic conditions | never constitutive) |
| COX-2 | Most colorectal carcinomas; | Cells at sites of inflammation |
| | many lung carcinomas; | |
| | possibly many other cancers | |
| IL-10 | Most colorectal carcinomas; | Leukocytes |
| | many lung carcinomas; many | |
| | squamous cell carcinomas of | |
| | head and neck; possibly many | |
| | other cancers | |
| GRP78/BiP | Presumably many different | Cells at sites of ishemia |
| | cancers, since GRP7S | |
| | expression is induced by | |
| | tumor-specific conditions | |
| CarG elements from Egr-1 | Induced by ionization | Cells exposed to ionizing |
| | radiation, so conceivably most | radiation; leukocytes |
| | tumors upon irradiation | |

A specific initiation signal also may be required for efficient translation of coding, sequences. These signals include the ATG initiation codon or adjacent sequences, Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

2. IRES

In certain embodiments of the invention, the use of internal ribosome entry sites (IRES) elements are used to create multigene, or polycistronic, messages. IRES elements arc able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988) as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, herein incorporated by reference).

3. Multi-Purpose Cloning Sites

Vectors can include a multiple cloning site (MCS), which is a nucleic acid region that contains multiple restriction enzyme sites, any of which can be used in conjunction with standard recombinant technology to digest the vector. See Carbonelli et al., 1999. Levenson et al., 1998, and Cocea, 1997, incorporated herein by reference. “Restriction enzyme digestion” refers to catalytic cleavage of a nucleic acid molecule with an enzyme that functions only at specific locations in a nucleic and molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Spiking Sites

Most transcribed eukaryotic RNA molecules will undergo RNA splicing to remove introns from the primary transcripts. Vectors containing genomic eukaryotic sequences may require donor and/or acceptor splicing Sites to ensure proper processing of the transcript for protein expression (see Chandler et al., 1997, herein incorporated by reference).

5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport,

7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

8. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygyomycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

9. Viral Vectors

The capacity of certain viral vectors to efficiently infect or enter cells, to integrate into a host cell genome and stably express viral genes, have led to the development and application of a number of different viral vector systems (Robbins et al., 1998), Viral systems are currently being developed for use as vectors for ex vivo and in vivo gene transfer. For example, adenovirus, herpes-simplex virus, retrovirus and adeno-associated virus vectors are being evaluated currently for treatment of diseases such as cancer, cystic fibrosis, Gaucher disease, renal disease and arthritis (Robbins and Ghivizzani, 1998; Imai et al., 1998; U.S. Pat. No. 5,670,488). The various viral vectors described below, present specific advantages and disadvantages, depending on the particular gene-therapeutic application.

Adenoviral Vectors.

In particular embodiments, an adenoviral expression vector is contemplated for the delivery of expression constructs. “Adenovirus expression vector” is meant to include those constructs containing adenovirus sequences sufficient to (a) support packaging of the construct and (b) to ultimately express a tissue or cell-specific construct that has been cloned therein.

Adenoviruses comprise linear, double-stranded DNA, with a genome ranging from 30 to 35 kb in size (Reddy et al., 1998; Morrison et al., 1997; Chilton et al., 1999), An adenovirus expression vector according to the present invention comprises a genetically engineered form of the adenovirus. Advantages of adenoviral gene transfer include the ability to infect a wide variety of cell types, including non-dividing cells, a mid-sized genome, ease of manipulation, high infectivity and the ability to be grown to high titers (Wilson, 1996). Further, adenoviral infection of host cells does not result in chromosomal integration because adenoviral DNA can replicate in an episomal manner, without potential genotoxicity associated with other viral vectors. Adenoviruses also are structurally stable (Marienfeld et al., 1999) and no genome rearrangement has been detected after extensive amplification (Parks et al., 1997; Bett et al., 1993).

Salient features of the adenovirus genome are an early region (E1, E2, E3 and E4 genes), an intermediate region (pIX gene, Iva2 gene), a late region (L1, L2, L3, L4 and L5 genes), a major late promoter (MLP), inverted-terminal-repeats (TTRs) and ψ sequence (Zheng, et al., 1999; Robbins et al., 1998; Graham and Prevec, 1995). The early genes E1, E2, E3 and E4 are expressed from the virus after infection and encode polypeptides that regulate viral gene expression, cellular gene expression, viral replication, and inhibition of cellular apoptosis. Further on during viral, infection, the MLP is activated, resulting in the expression of the late (L) genes, encoding polypeptides required for adenovirus eneapsidation. The intermediate region encodes components of the adenoviral capsid, Adenoviral inverted terminal repeats (ITRs; 100-200 bp in length), are cis elements, and function as origins of replication and are necessary for viral DNA replication. The ψ sequence is required for the packaging of the adenoviral genome.

A common approach for generating adenoviruses for use as a gene transfer vectors is the deletion of the E1 gene (E1⁻), which is involved in the induction of the E2, E3 and E4 promoters (Graham and Prevec, 1995). Subsequently, a therapeutic gene or genes can be inserted recombinantly in place of the E1 gene, wherein expression of the therapeutic gene(s) is driven by the E1 promoter or a heterologous promoter. The E1⁻, replication-deficient virus is then proliferated in a “helper” cell line that provides the E1 polypeptides in trans (e.g., the human embryonic kidney cell line 293). Thus, in the present invention it may be convenient to introduce the transforming construct at the position from which the E1-coding sequences have been removed. However, the position of insertion of the construct within the adenovirus sequences is not critical to the invention. Alternatively, the E3 region, portions of the E4 region or both may be deleted, wherein a heterologous nucleic acid sequence under the control of a promoter operable in eukaryotic cells is inserted into the adenovirus genome for use in gene transfer (U.S. Pat. No. 5,670,488; U.S. Pat. No. 5,932,210, each specifically incorporated herein by reference).

Although adenovirus based vectors offer several unique advantages over other vector systems, they often are limited by vector immunogenicity, size constraints for insertion of recombinant genes and low levels of replication. The preparation of a recombinant adenovirus vector deleted of all open reading frames, comprising a full length dystrophin gene and the terminal repeats required for replication (Haecker et al., 1996) offers some potentially promising advantages to the above mentioned adenoviral shortcomings. The vector was grown to high titer with a helper virus in 293 cells and was capable of efficiently transducing dystrophin in mdx mice, in myotubes in vitro and muscle fibers in vivo. Helper-dependent viral vectors are discussed below.

A major concern in using adenoviral vectors is the generation of a replication-competent virus during vector production in a packaging cell line or during gene therapy treatment of an individual. The generation of a replication-competent virus could pose serious threat of an unintended viral infection and pathological consequences for the patient. Armentano et al. (1990), describe the preparation of a replication-defective adenovirus vector, claimed to eliminate the potential for the inadvertent generation of a replication-competent adenovirus (U.S. Pat. No. 5,824,544, specifically incorporated herein by reference). The replication-defective adenovirus method comprises a deleted E1 region and a relocated protein IX gene, wherein the vector expresses a heterologous, mammalian gene.

Other than the requirement that the adenovirus vector be replication defective, or at least conditionally defective, the nature of the adenovirus vector is not believed to be crucial to the successful practice of the invention. The adenovirus may be of any of the 42 different known serotypes and/or subgroups A-F. Adenovirus type 5 of subgroup C is the preferred starting material in order to obtain the conditional replication-defective adenovirus vector for use in the present invention. This is because adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Adenovirus growth and manipulation is known to those of skiff in the art, and exhibits broad host range in vitro and in vivo (U.S. Pat. No. 5,670,488; U.S. Pat. No. 5,932,210; U.S. Pat. No. 5,824,544). This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. Many experiments, innovations, preclinical studies and clinical trials are currently under investigation fix the use of adenoviruses as gene delivery vectors. For example, adenoviral gene delivery-based gene therapies are being developed far liver diseases (Han et al., 1999), psychiatric diseases (Leach, 1999), neurological diseases (Smith, 1998; Hermens and Verhaagen, 1998), coronary diseases (Feldman et al., 1996), muscular diseases (Petrof, 1998), gastrointestinal diseases (Wu, 1998) and various cancers such as colorectal (Fujiwara and Tanaka, 1998; Dorai et al., 1999), pancreatic, bladder (Irie et al., 1999), head and neck (Blackwell et al., 1999), breast (Stewart et al., 1999), lung (Batra et al., 1999) and ovarian (Vanderkwaak et al., 1999).

Retroviral Vectors.

In certain embodiments of the invention, the uses of retroviruses for gene delivery are contemplated. Retroviruses are RNA viruses comprising an RNA genome. When a host cell is infected by a retrovirus, the genomic RNA is reverse transcribed into a DNA intermediate which is integrated, into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

The retroviral genome and the proviral DNA have three genes: gag, poi, and env, which are flanked by two long terminal repeat (LTR) sequences. The gag gene encodes the internal structural (matrix, capsid, and nueleorapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication.

A recombinant retrovirus of the present invention may be genetically modified in such a way that some of the structural, infectious genes of the native virus have been removed and replaced instead with a nucleic acid sequence to be delivered, to a target cell (U.S. Pat. No. 5,858,744; U.S. Pat. No. 5,739,018, each incorporated herein by reference). After infection of a cell by the virus, the virus injects its nucleic acid into the cell and the retrovirus genetic material can integrate into the host cell genome. The transferred retrovirus genetic material is then transcribed and translated into proteins within the host cell. As with other viral vector systems, the generation of a replication-competent retrovirus during vector production or during therapy is a major concern. Retroviral vectors suitable for use in the present invention are generally defective retroviral vectors that are capable of infecting the target cell, reverse transcribing their RNA genomes, and integrating the reverse transcribed DNA into the target cell genuine, but are incapable of replicating within the target cell to produce infectious retroviral particles (e.g., the retroviral genuine transferred into the target cell is defective in gag, the gene encoding virion structural proteins, and/or in poi, the gene encoding reverse transcriptase). Thus, transcription of the provirus and assembly into infectious virus occurs in the presence of an appropriate helper virus or in a cell line containing appropriate sequences enabling encapsidation without coincident production of a contaminating helper virus.

The growth and maintenance of retroviruses is known in the art (U.S. Pat. No. 5,955,331; U.S. Pat. No. 5,888,502, each specifically incorporated herein by reference). Nolan et al. describe the production of stable high titre, helper-free retrovirus comprising a heterologous gene (U.S. Pat. No. 5,830,725, specifically incorporated herein by reference). Methods for constructing packaging cell lines useful for the generation of helper-free recombinant retroviruses with amphoteric or ecotrophic host ranges, as well as methods of using the recombinant retroviruses to introduce a gene of interest into eukaryotic cells in vivo and in vitro are contemplated in the present invention (U.S. Pat. No. 5,955,331).

Currently, the majority of all clinical trials for vector-mediated gene delivery use murine leukemia virus (MLV)-based retroviral vector gene delivery (Robbins et al., 1998; Miller et al., 1993). Disadvantages of retroviral gene delivery include a requirement for ongoing cell division for stable infection and a coding capacity that prevents the delivery of large genes. However, recent development of vectors such as lentivirus (e.g., HIV), simian immunodeficiency virus (SIV) and equine infectious-anemia virus (EIAV), which can infect certain non-dividing cells, potentially allow the in rive, use of retroviral vectors for gene therapy applications (Amado and Chen, 1999, Klimatcheva et al., 1999; White et al., 1999; Case et al., 1999). For example, HIV-based vectors have been used to infect non-dividing cells such as neurons (Miyatake et al., 1999), islets (Leibowitz et al., 1999) and muscle cells (Johnston et al., 1999). The therapeutic delivery of genes via retroviruses are currently being assessed for the treatment of various disorders such as inflammatory disease (Moldawer et al., 1999), AIDS (Amado and Chen, 1999; Engel and Kohn, 1999), cancer (Clay et al., 1999), cerebrovascular disease (Weihl et al., 1999) and hemophilia (Kay, 1998).

Herpesviral Vectors.

Herpes simplex virus (HSV) type I and type II contain a double-stranded, linear DNA genome of approximately 150 kb, encoding 70-80 genes, Wild type HSV are able to infect cells lytically and to establish latency in certain cell types (e.g., neurons). Similar to adenovirus, HSV also can infect a variety of cell types including muscle (Yeung et al., 1999), ear (Derby et al., 1990), eye (Kaufman et al., 1999), tumors (Yoon at al., 1999; Howard et al., 1999), lung (Kohut et al., 1998), neuronal (Garrido et al., 1999; Lachmann and Efstathiou, 1999), liver (Miytake et al., 1999; Kooky et al., 1999) and pancreatic islets (Rabinovitch et al., 1999).

HSV viral genes are transcribed by cellular RNA polymerase II and are temporally regulated, resulting in the transcription and subsequent synthesis of gene products in roughly three discernable phases or kinetic classes. These phases of genes are referred to as the Immediate Early (IE) or α genes, Early (E) or β genes and Late (L) or γ genes. Immediately following the arrival of the genome of a virus in the nucleus of a newly infected cell, the IE genes are transcribed. The efficient expression of these genes does not require prior viral protein synthesis. The products of IE genes arc required to activate transcription and regulate the remainder of the viral genome.

Ear use in therapeutic gene delivery, HSV must be rendered replication-defective. Protocols for generating replication-defective HSV helper virus-free cell lines have been described (U.S. Pat. No. 5,879,934; U.S. Pat. No. 5,851,826, each specifically incorporated herein by reference in its entirety). One IE protein, ICP4, also known as α4 or Vmw175, is absolutely required for both virus infectivity and the transition from IE to later transcription. Thus, due to its complex, multifunctional nature and central rote in the regulation of HSV gene expression. ICP4 has typically been the target of HSV genetic studies.

Phenotypic studies of HSV viruses deleted of ICP4 indicate that such viruses will be potentially useful for gene transfer purposes (Krisky et al., 1998a). One property of viruses deleted for ICP4 that makes them desirable for gene transfer is that they only express the five other IE genes: ICP0, ICP6, ICP27, ICP22 and ICP47 (DeLuca et al., 1985), without the expression of viral genes encoding proteins that direct viral DNA synthesis, as well as the structural proteins of the virus. This property is desirable for minimizing possible deleterious effects on host cell metabolism or an immune response following gene transfer. Further deletion of IE genes ICP22 and ICP27, in addition to ICP4, substantially improve reduction of HSV cytotoxicity and prevented early and late viral gene expression (Krisky et al., 1998b).

The therapeutic potential of HSV in gene transfer has been demonstrated in various in vitro model systems and in vivo for diseases such as Parkinson's (Yamada et al., 1999), retinoblastoma (Hayashi at 1999), intracerebral and intradermal tumors (Moriuchi et al., 1998), B-cell malignancies (Suzuki et al., 1998), ovarian cancer (Wang et al., 1998) and Duchenne muscular dystrophy (Huard et al., 1997).

Adeno-Associated Viral Vectors.

Adeno-associated virus (AAV), a member of the parvovirus family, is a human virus that is increasingly being used for gene delivery therapeutics. AAV has several advantageous features not found in other viral systems. First, AAV can infect a wide range of host cells, including non-dividing cells. Second. AAV can infect cells from different species. Third. AAV has not been associated with any human or animal, disease and does not appear to alter the biological properties of the host cell upon integration. For example, it is estimated that 80-85% of the human population has been exposed to AAV. Finally, AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. The internal non-repeated portion of the genome includes two large open reading frames, known as the AAV replication (rep) and capsid (cap) genes. The rep and cap genes code for viral proteins that allow the virus to replicate and package the viral genome into a virion. A family of at least four viral proteins is expressed from the AAV rep region. Rep 78Rep 68, Rep 52, and Rep 40, named according to their apparent molecular weight. The AAV cap region encodes at least three proteins, VP1, VP2, and VP3.

AAV is a helper-dependent virus requiring c infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genuine into infectious AAV virions. Although AAV can infect cells from different species, the helper virus must be of the same species as the host cell (e.g., human AAV will replicate in canine cells co-infected with a canine adenovirus).

AAV has been engineered to deliver genes of interest by deleting the internal non-repeating portion of the AAV genome and inserting a heterologous gene between the ITRs. The heterologous gene may be functionally linked to a heterologous promoter (constitutive, cell-specific, or inducible) capable of driving gene expression in target cells. To produce infectious recombinant AAV (rAAV) containing a heterologous gene, a suitable producer cell line is transfected with a rAAV vector containing a heterologous gene. The producer cell is concurrently transfected with a second plasmid harboring the AAV rep and cap genes under the control of their respective endogenous promoters or heterologous promoters. Finally, the producer cell is infected with a helper virus.

Once these factors come together, the heterologous gene is replicated and packaged as though it were a wild-type AAV genome. When target cells are infected with the resulting rAAV virions, the heterologous gene enters and is expressed in the target cells. Because the target cells lack the rep and cap genes and the adenovirus helper genes, the rAAV cannot further replicate, package or form wild-type AAV.

The use of helper virus, however, presents a number of problems. First, the use of adenovirus in a rAAV production system causes the host cells to produce both rAAV and infectious adenovirus. The contaminating infectious adenovirus can be inactivated by heat treatment (56° C. for 1 hour). Heat treatment, however, results in approximately a 50% drop in the titer of functional rAAV virions. Second, varying amounts of adenovirus proteins are present in these preparations. For example, approximately 50% or greater of the total protein obtained in such rAAV virion preparations is free adenovirus fiber protein. If not completely removed, these adenovirus proteins have the potential of eliciting an immune response from the patient. Third. AAV vector production methods which employ a helper virus require the use and manipulation of large amounts of high titer infectious helper virus, which presents a number of health and safety concerns, particularly in regard to the use of a herpesvirus. Fourth, concomitant production of helper virus particles in rAAV virion producing cells diverts large amounts of host cellular resources away from rAAV virion production, potentially resulting in lower rAAV virion yields.

Lentiviral Vectors.

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human immunodeficiency Viruses: HIV-1, HIV-2 and the Simian immunodeficiency Virus: SIV. Lentiviral vectors have been generated by multiply attenuating the HIV virulence genes, for example, the genes env, vif vpr, vpu and nef are deleted making the vector biologically safe.

Recombinant lentiviral vectors are capable of infecting non-dividing cells and can be used for both in vivo and ex vivo gene transfer and expression of nucleic acid sequences. The lentiviral genome and the pro viral DNA have the three genes found in retroviruses: gag, pol and env, which arc flanked by two long terminal repeat (LTR) sequences. The gag gene encodes the internal structural (matrix, capsid and nucleocapsid) proteins; the poi gene encodes the RNA-directed DNA polymerase (reverse transcriptase), a protease and an integrase; and the env gene encodes viral envelope glycoprotens. The 5′ and 3′ LTR's serve, to promote transcription and polyadenylation of the virion RNA's. The LTR contains all other cis-acting sequences necessary for viral replication. Lentiviruses have additional genes including vif vpr, tat, rev, vpu, nef and vpx.

Adjacent to the 5′ LTR are sequences necessary for reverse transcription of the genome (the tRNA primer binding site) and for efficient encapsidation of viral RNA into particles the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

Lentiviral vectors are known in the art, see Naldini et al., (1996); Zufferey et al., (1997); U.S. Pat. Nos. 6,013,516; and 5,994,136, in general, the vectors are plasmid-based or virus-based, and are configured to carry the essential sequences for incorporating foreign nucleic acid, for selection and for transfer of the nucleic acid into a host cell. The gag, pol and ear genes of the vectors of interest also are known in the art. Thus, the relevant genes arc cloned into the selected vector and then used to transform the target cell of interest.

Recombinant lentivirus capable of infecting a non-dividing cell wherein a suitable host cell is transfected with two or more vectors carrying the packaging functions, namely gag, poi and env, as well as rev and tat is described in U.S. Pat. No. 5,994,136, incorporated herein by reference. This describes a first vector that can provide a nucleic acid encoding a viral gag and a pal gene and another vector that can provide a nucleic acid encoding a viral env to produce a packaging cell. Introducing a vector providing a heterologous gene, such as the STAT-1α gene in this invention, into that packaging cell yields a producer cell which releases infectious viral particles carrying the foreign gene of interest. The env preferably is an amphotropic envelope protein which allows transduction of cells of human and other species.

One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

The vector providing the viral env nucleic acid sequence is associated operably with regulatory sequences, e.g., a promoter or enhancer. The regulatory sequence can be any eukaryotic promoter or enhancer, including for example, the Moloney marine leukemia virus promoter-enhancer element, the human cytomegalovirus enhancer or the vaccinia P7.5 promoter. In some cases, such as the Moloney murine leukemia virus promoter-enhancer element, the promoter-enhancer elements are located within or adjacent to the LTR sequences.

The heterologous or foreign nucleic acid sequence, such as the STAT-1α encoding polynucleotide sequence herein, is linked operably to a regulatory nucleic acid sequence. Preferably, the heterologous sequence is linked to a promoter, resulting in a chimeric gene. The heterologous nucleic acid sequence may also be under control of either the viral LTR promoter-enhancer signals or of an internal promoter, and retained signals within the retroviral LTR can still bring about efficient expression of the transgene. Marker genes may be utilized to assay for the presence of the vector, and thus, to confirm infection and integration. The presence of a marker gene ensures the selection and growth of only those host cells which express the inserts. Typical selection genes encode proteins that confer resistance to antibiotics and other toxic substances, cg, histidinol, puromycin, hygromycin, neomycin, methotrexate, etc., and cell surface markers.

The vectors are introduced via transfection or infection into the packaging cell line. The packaging cell line produces viral particles that contain the vector genome. Methods for transfection or infection are well known by those of skill in the art. After cotransfection of the packaging vectors and the transfer vector to the packaging cell line, the recombinant virus is recovered from the culture media and titered by standard methods used by those of skill in the art. Thus, the packaging constructs can be introduced into human cell lines by calcium phosphate transfection, lipofection or electroporation, generally together with a dominant selectable marker, such as neo, DHFR, Gln synthetase or ADA, followed by selection in the presence of the appropriate drug and isolation of clones. The selectable marker gene can be linked physically to the packaging genes in the construct.

Lentiviral transfer vectors Naldini et al. (1996), have been used to infect human cells growth-arrested in vitro and to transduce neurons after direct injection into the brain of adult rats. The vector was efficient at transferring marker genes in vivo into the neurons and long term expression in the absence of detectable pathology was achieved. Animals analyzed ten months after a single injection of the vector showed no decrease in the average level of transgene expression and no sign of tissue pathology or immune reaction (Blomer et al., 1997): Thus, in the present invention, one may graft or transplant cells infected with the recombinant lentivirus ex vivo, or infect cells in vivo.

Other Viral Vectors.

The development and utility of viral vectors for gene delivery is constantly improving and evolving. Other viral vectors such as poxvirus; e.g., vaccinia virus (Guant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present invention and may be selected according to the requisite properties of the target system.

In certain embodiments, vaccinia viral vectors are contemplated for use in the present invention. Vaccinia virus is a particularly useful eukaryotic viral vector system for expressing heterologous genes. For example, when recombinant vaccinia virus is properly engineered, the proteins are synthesized, processed and transported to the plasma membrane. Vaccinia viruses as gene delivery vectors have recently been demonstrated to transfer genes to human tumor cells, e.g., EMAP-II (Gnant et al., 1999), inner ear (Derby et al., 1999), glioma cells, e.g., p53 (Timiryasova et al., 1999) and various mammalian cells, e.g. P₄₅₀ (U.S. Pat. No. 5,506,138). The preparation, growth and manipulation of vaccinia viruses are described in U.S. Pat. No. 5,849,304 and U.S. Pat. No. 5,506,138 (each specifically incorporated herein by reference).

In other embodiments, sindbis viral vectors are contemplated for use in gene delivery. Sindbis virus is a species of the alphavirus genus (Garoff and Li, 1998) which includes such important pathogens as Venezuelan. Western and Eastern equine encephalitis viruses (Sawai et al., 1999; Mastrangelo et al., 1999), in vitro, sindbis virus infects a variety of avian, mammalian, reptilian, and amphibian cells. The genome of sindbis virus consists of a single molecule of single-stranded RNA, 11,703 nucleotides in length. The genomic RNA is infectious, is capped at the 5′ terminus and polyadenylated at the 3 terminus, and serves as mRNA. Translation of a vaccinia virus 26S mRNA produces a polyprotein that is cleaved co- and post-translationally by a combination of viral and presumably host-encoded proteases to give the three virus structural proteins, a capsid protein (C) and the two envelope glycoproteins (E1 and PE2, precursors of the virion E2).

Three features of sindbis virus suggest that it would be a useful vector for the expression of heterologous genes. First, its wide host range, both in nature and in the laboratory. Second, gene expression occurs in the cytoplasm of the host cell and is rapid and efficient. Third, temperature-sensitive mutations in RNA synthesis are available that may be used to modulate the expression of heterologous coding sequences by simply shifting cultures to the non-permissive temperature at various time after infection. The growth and maintenance of sindbis virus is known in die art (U.S. Pat. No. 5,217,879, specifically incorporated herein by reference).

Chimeric Viral Vectors.

Chimeric or hybrid viral vectors are being developed for use in therapeutic gene delivery and are contemplated for use in the present invention. Chimeric pox viral/retroviral vectors (Holzer et al., 1999), adenoviral/retroviral vectors (Feng 1997; Bilbao et al., 1997; Caplen et al., 1999) and adenoviral/adeno-associated viral vectors (Fisher et al., 1996; U.S. Pat. No. 5,871,982) have been described.

These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. No. 5,871,983, specifically incorporate herein by reference).

The adenovirus/AAV chimeric virus uses adenovirus nucleic acid sequences as a shuttle to deliver a recombinant AAV/transgene genome to a target cell. The adenovirus nucleic acid sequences employed in the hybrid vector can range from a minimum sequence amount, which requires the use of a helper virus to produce the hybrid virus particle, to only selected deletions of adenovirus genes, which deleted gene products cats be supplied in the hybrid viral production process by a selected packaging cell. At a minimum, the adenovirus nucleic acid sequences employed in the pAdA shuttle vector are adenovirus genomic sequences from which all viral genes are deleted and which contain only those adenovirus sequences required for packaging adenoviral genomic DNA into as preformed capsid head. More specifically, the adenovirus sequences employed are the cis-acting 5′ and 3′ inverted terminal repeat (ITR) sequences of an adenovirus (which function as origins of replication) and the native 5 packaging/enhancer domain, that contains sequences necessary for packaging linear Ad genomes and enhancer elements for the E1 promoter. The adenovirus sequences may be modified to contain desired deletions, substitutions, or mutations, provided that the desired function is not eliminated.

The AAV sequences useful in the above chimeric vector are the viral sequences from which the rep and cap polypeptide encoding sequences are deleted. More specifically, the AAV sequences employed are the cis-acting 5′ and 3′ inverted terminal repeat (ITR) sequences. These chimeras are characterized by high titer transgene delivery to a host cell and the ability to stably integrate the transgene into the host cell chromosome (U.S. Pat. No. 5,871,983, specifically incorporate herein by reference). In the hybrid vector construct, the AAV sequences are flanked by the selected adenovirus sequences discussed above. The 5′ and 3′ AAV ITR sequences themselves flank a selected transgene sequence and associated regulatory elements, described below. Thus, the sequence formed by the transgene and flanking 5′ and 3′ AAV sequences may be inserted at any deletion site in the adenovirus sequences of the vector. For example, the AAV sequences are desirably inserted at the site of the deleted E1 a/E1b genes of the adenovirus. Alternatively, the AAV sequences may be inserted at an E3 deletion, E2a deletion, and so on. If only the adenovirus 5′ ITR/packaging sequences and 3′ ITR sequences are used in the hybrid virus, the AAV sequences are inserted between them.

The transgene sequence of the vector and recombinant virus can be a gene, a nucleic acid sequence or reverse transcript thereof, heterologous to the adenovirus sequence, which encodes a protein, polypeptide or peptide fragment of interest. The transgene is operatively linked to regulatory components in a manner which permits transgene transcription. The composition of the transgene sequence will depend upon the use to which the resulting hybrid vector will be put. For example, one type of transgene sequence includes a therapeutic gene which expresses a desired gene product in a host cell. These therapeutic genes or nucleic acid sequences typically encode products for administration and expression in a patient in vivo or ex vivo to replace or correct an inherited or non-inherited genetic defeat or treat an epigenetic disorder or disease.

10. Non-Viral Transformation

Suitable methods for nucleic acid delivery for transformation of an organelle, a cell, a tissue or an organism for use with the current invention arc believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organclle(s), cell(s), tissue(s) or organism(s) may be stably or transiently transformed.

Injection.

In certain embodiments, a nucleic acid may be delivered to an organelle, a cell, a tissue or an organism via one or more injections (i.e., a needle injection), such as, for example, either subcutaneously, intradermally, intramuscularly, intervenously or intraperitoneally. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oncytes (Harland and Weintraub, 1985).

Electroporation.

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes. such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human κ-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyase) or mechanically wounding, in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hon and Lin, 1996), soybean (Christen et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 92/17598, incorporated herein b reference. Other examples of species thr which protoplast transformation has been described include barley (Lazerri, 1995), sorghum (et al., 1991), maize (Bhattacharjee et al., 1997), wheat (He et al., 1994) and tomato (Tsukada, 1989).

Calcium Phosphate.

In other embodiments of the present invention, a nucleic acid is introduced to the cells using calcium phosphate precipitation, Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

DEAE-Dextran: In another embodiment, a nucleic acid is delivered into a cell using DEAE-dextran followed by polyethylene glycol. In this manner, reporter plasmids were introduced into mouse in myeloma and erythroleukemia cells (Gopal, 1985).

Sonication Loading.

Additional embodiments of the present invention include the introduction of a nucleic acid by direct sonic loading. LTK fibroblasts have been transfected with the thymidine kinase gene by sonication loading (Fechheimer et al., 1987).

Liposome-Mediated Transfection.

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghost and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicola et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

Receptor-Mediated Transfection.

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the BCE receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

11. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MaxBac® 2.0 from Invitrogen® and BacPack™ Baculovirus Expression System From Clontech®.

Other examples of expression systems include Stratagene®'s Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and tell lines of Chinese hamster ovary, W138, BHK, COS-7, 293, HepG2, NIH3T3, RIN and MDCK cells. In addition, a host cell strain may be chosen that modulates the expression of the inserted, sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti metabolite resistance can be used as the basis of selection for dhfr, that confers resistance to; gpt, that confers resistance to myeophenolic acid; neo, that confers resistance to the aminoglycoside G418; and hygro, that confers resistance to hygromyein.

D. Design, Variants and Analogs

Variants of the peptide/polypeptide sequences discussed above may be employed. For example, certain non-natural amino acids that satisfy the structural constraints of the ligand traps may be substituted without a loss, and perhaps with an improvement in, biological function. In addition, the present inventors also contemplate that structurally similar compounds May be formulated to mimic the key portions of peptide, or polypeptides of the present invention. Such compounds, which May be termed peptidomimetics, may be used in the same manner as the peptides of the invention and hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Methods for generating specific structures have been disclosed in the art. For example, α-helix mimetics are disclosed in U.S. Pat. Nos. 5,446,128; 5,710,245; 5,840,833; and 5,859,184. Methods for generating conformationally restricted β-turns and β-bulges are described, for example, in U.S. Pat. Nos. 5,440,013; 5,618,914; and 5,670,155. Other types of mimetic turns include reverse and γ-turns. Reverse turn mimetics are disclosed in U.S. Pat. Nos. 5,475,085 and 5,929,237, and γ-turn mimetics are described in U.S. Pat. Nos. 5,672,681 and 5,674,976.

By “molecular modeling” is meant quantitative and/or qualitative analysis of the structure and function of protein-protein physical interaction based on three-dimensional structural information and protein-protein interaction models. This includes conventional numeric-based molecular dynamic and energy minimization models, interactive computer graphic models, modified molecular mechanics models, distance geometry and other structure-based constraint models. Molecular modeling typically is performed using a computer and may be further optimized using known methods. Computer programs that use X-ray crystallography data are particularly useful for designing such compounds. Programs such as RasMol, for example, ran be used to generate three dimensional models. Computer programs such as INSIGHT (Accelrys, Burlington, Mass.), GRASP (Anthony Nicholls, Columbia University), Dock (Molecular Design Institute, University of California at San Francisco), and Auto-Dock (Accelrys) allow for further manipulation and the ability to introduce new structures. The methods can involve the additional step of outputting to an output device a model of the 3-D structure of the compound. In addition, the 3-D data of candidate compounds can be compared to a computer database of, for example, 3-D structures.

Compounds of the invention also may be interactively designed from structural information of the compounds described herein using other structure-based design/modeling techniques (see, e.g., Jackson, 1997; Jones et al., 1996), Candidate compounds can then be tested in standard assays familiar to those skilled in the art. Exemplary assays are described herein.

The 3-D structure of biological macromolecules (e.g., proteins, nucleic acids, carbohydrates, and lipids) can be determined from data obtained by a variety of methodologies. These methodologies, which have been applied most effectively to the assessment of the 3-D structure of proteins, include: (a) x-ray crystallography; (b) nuclear magnetic resonance (NMR) spectroscopy; (c) analysis of physical distance constraints formed between defined sites on a macromolecule, e.g., intramolecular chemical crosslinks between residues on a protein (e.g., PCT/US00/14667, the disclosure of which is incorporated herein by reference in its entirety), and (d) molecular modeling methods based on a knowledge of the primary structure of a protein of interest, e.g., homology modeling techniques, threading algorithms, or ab initio structure modeling using computer programs such as MONSSTER (Modeling Of New Structures from Secondary and Tertiary Restraints) (see, e.g., International Application No. PCT/US99/11913, the disclosure of which is incorporated herein by reference in its entirety). Other molecular modeling techniques may also be employed in accordance with this invention (e.g., Cohen et al., 1990; Navia et al., 1992), the disclosures of which are incorporated herein by reference in their entirety). All these methods produce data that are amenable to computer analysis. Other spectroscopic methods that can also be useful in the method of the invention, but that do not currently provide atomic level structural detail about biomolecules, include circular dichroism and fluorescence and ultraviolet/visible light absorbance spectroscopy. A preferred method of analysis is x-ray crystallography. Descriptions of this procedure and of NMR spectroscopy are provided below.

X-Ray Crystallography.

X-ray crystallography is based on the diffraction of x-radiation of a characteristic wavelength by electron clouds surrounding the atomic nuclei in a crystal of a molecule or molecular complex of interest. The technique uses crystals of purified biological macromolecules or molecular complexes (hut these frequently include solvent components, co-factors, substrates, or other ligands) to determine near atomic resolution of the atoms ranking up the particular biological macromolecule. A prerequisite for solving 3-D structure by x-ray crystallography is a well-ordered crystal that will diffract x-rays strongly. The method directs a beam of x-rays onto a regular, repeating array of many identical molecules so that the x-rays are diffracted from the array in a pattern from which the structure of an individual molecule can be retrieved. Well-ordered crystals of, for example, globular protein molecules are large, spherical or ellipsoidal objects with irregular surfaces. The crystals contain, large channels between the individual molecules. These channels, which normally occupy more than one half the volume of the crystal, are filled with disordered solvent molecules, and the protein molecules are in contact with each ether at only a few small regions. This is one reason why structures of proteins in crystals are generally the same as those of proteins in solution.

In diffraction experiments, a narrow and parallel beam of x-rays is taken from the x-ray source and directed onto the crystal to produce diffracted beams. The incident primary beams cause damage to both the macromolecule and solvent molecules. The crystal, is, therefore, cooled (e.g., to between −220° C. and −50° C.) to prolong its lifetime. The primary beam must strike the crystal from many directions to produce all possible diffraction spots, so the crystal is rotated in the beam during the experiment. The diffracted spots are recorded on a film or by an electronic detector. Exposed film has to be digitized and quantified in a scanning device, whereas the electronic detectors feed the signals they detect directly into a computer. Electronic area detectors significantly reduce the time required to collect and measure diffraction data. Each diffraction beam, which is recorded as a spot cm film or a detector plate, is defined by three properties: the amplitude, which is measured from the intensity of the spot; the wavelength, which is set by the x-ray source; and the phase, which is lost in x-ray experiments. All three properties are needed for all of the diffracted beams in order to determine the positions of the atoms giving rise to the diffracted beams. One way of determining the phases is called Multiple Isomorphous Replacement (MIR), which requires the introduction of exogenous x-ray scatterers (e.g., heavy atoms such metal atoms) into the unit cell of the crystal. For a more detailed description of MIR, see U.S. Pat. No. 6,093,573 (column 15) the disclosure of which is incorporated herein by reference in its entirety.

Atomic coordinates refer to Cartesian coordinates (x, y, and z positions) derived from mathematical equations involving Fourier synthesis of data derived from patterns obtained via diffraction of a monochromatic beam of x-rays by the atoms (scattering centers) of biological macromolecule of interest in crystal form. Diffraction data are used to calculate electron density maps of repeating units in the crystal (unit cell), Electron density maps are used to establish the positions (atomic coordinates) of individual atoms within a crystal's unit cell. The absolute values of atomic coordinates convey spatial relationships between atoms because the absolute values ascribed to atomic coordinates can be changed by rotational and/or translational movement along x, y, and/or z axes, together or separately, while maintaining the same relative spatial relationships among atoms. Thus, a biological macromolecule (e.g., a protein) whose set of absolute atomic coordinate values can be rotationally or translationally adjusted to coincide with a set of prior determined values from an analysis of another sample is considered to have the same atomic coordinates as those obtained from the other sample.

Further details on x-ray crystallography can be obtained from co-pending U.S. Application No. 2005/0015232, U.S. Pat. No. 6,093,573 and International Application Nos. PCT/US99/18441, PCT/US99/11913, and PCT/US00/03745. The disclosures of all these patent documents are incorporated herein by reference in their entirety.

NMR Spectroscopy.

Whereas x-ray crystallography requires single crystals of a macromolecule of interest, NMR measurements are carried out in solution under near physiological conditions. However, NMR-derived structures are not as detailed as crystal-derived structures.

While the use of NMR spectroscopy was until relatively recently limited to the elucidation of the 3-D structure of relatively small molecules (e.g., proteins of 100-150 amino acid residues), recent advances including isotopic labeling of the molecule of interest and transverse relaxation-optimized spectroscopy (TROSY) have allowed the methodology to be extended to the analysis of much larger molecules, e.g., proteins with a molecular weight of 110 kDa (Wider, 2000).

NMR uses radio-frequency radiation to examine the environment of magnetic atomic nuclei in a homogeneous magnetic field pulsed with a specific radio frequency. The pulses perturb the nuclear magnetization of those atoms with nuclei of nonzero spin. Transient time domain signals are detected as the system returns to equilibrium. Fourier transformation of the transient signal into a frequency domain yields a one-dimensional NMR spectrum. Peaks in these spectra represent chemical shifts of the various active nuclei. The chemical shift of an atom is determined by its local, electronic environment, Two-dimensional NMR experiments can provide information about the proximity of Various atoms in the structure and in three dimensional space. Protein structures can be determined by performing a number of two- (and sometimes 3- or 4-) dimensional NMR experiments and using the resulting information as constraints in a series of protein folding simulations.

More information on NMR spectroscopy including detailed descriptions of how raw data obtained from an NMR experiment can be used to determine the 3-D structure of a macromolecule can be found in: Protein NMR Spectroscopy, Principles and Practice, (1996); Gronenborn et al. (1990); and Wider (2000), supra., the disclosures of all of which are incorporated herein by reference in their entirety

E. Peptoids/Peptidomimeties

In accordance with the present invention, peptoids are provided that mimic the native structure of the molecules discussed above. In general, one creates an achiral peptoid by moving the amino acid sidechains over to the amide nitrogen. For chemical stability reasons, cysteine and serine residues need to be homologated. Other residue side chain lengths can remain as is or be homologated in order to assure proper alignment relative to the native peptide based on modeling experiments. Sometimes, chirality is introduced into one of these side chains to induce a handedness to an alpha-helix secondary structure, although the peptides described here do not include such a strategy. Also, certain residues favor helix formation while others may not, and since it has been reported that the alpha-helix of peptoids turns a somewhat more tightly than normal petides, it has also been reported that some non-essential side chain residues must be deleted in order to maintain proper alignment of essential residues.

Also of interest are peptidomimetic compounds that are designed based upon the amino acid sequences of compounds of the invention that are peptides. Peptidomimetic compounds are synthetic compounds having a three-dimensional conformation “motif” that is substantially the same as the three-dimensional conformation of a selected peptide. Peptidomimetic compounds can have additional characteristics that enhance their in vivo utility, such as increased cell permeability and prolonged biological half-life. The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbonyl, dimethylene and ketomethylene bonds, are known in the art to be generally useful substitutes for peptide bonds in the construction of protease-resistant peptidomimetics.

Peptidomimetics are described in U.S. Pat. Nos. 5,939,268, 6,946,542, 7,166,568, 7,247,701, 7,589,170, 7,718,598, 7,863,239, 7,705,118, 7,202,332, 6,846,805, 6,706,862, 6,664,372, 6,566,493, 6,436,697, 6,197,963, 6,117,974, 5,817,879, 5,811,515, 5,811,512, 5,770,732, 5,552,534, 5,550,251, 5,288,707, and 5,250,564.

General design and methods of peptoid synthesis are described in U.S. Pat. Nos. 5,264,419, 5,801,148, 5,807,829, 5,811,387, 5,861,380, 5,869,455, 5,877,578 and 5,965,695, and U.S. Patent Publication 2003/0187188, 2005/0043509, which are exemplary in nature and incorporated herein by reference.

F. Purification of Proteins

It may be desirable to purify polypeptides according to the present invention. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete, purification (or purification to homogeneity). Analytical methods particularly suited to the preparation of a pure peptide rite ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein of peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein of peptide forms the major component of the composition, such as constituting about 50%, about 60%, about 70%. about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique Chosen to follow the purification and whether or not the expressed protein or peptide exhibits a detectable activity.

Various techniques suitable for use in protein purification will be well known to those of skill in the art. These include, for example, precipitation with ammonium sulphate. PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are elute from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is as chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography, Lectins are a class of substances that bind to a variety of polysaccharides and glycoproteins. Lectins arc usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fuctose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immumoaffinity chromatography. The generation of antibodies that would be suitable for use in accord with the present invention is discussed below.

### IV. THERAPIES

A. Pharmaceutical Formulations and Routes of Administration

Where clinical applications are contemplated, u will be necessary to prepare pharmaceutical compositions comprising the ligand traps in a form appropriate for the intended application. Generally, this will entail preparing compositions that are essentially free of pyrogens, as well as other impurities that could be harmful to humans or animals.

One will generally desire to employ appropriate salts and buffers to render the MUC1 ligand traps molecule or deliver vectors therefor stable and allow for uptake by target cells. Aqueous compositions of the present invention comprise an effective amount of the polypeptide/vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art, Except insofar as any conventional media or agent is incompatible with the vectors or polypeptides of the present invention, its use in therapeutic compositions is contemplated. Supplementary active ingredients also can be incorporated into the compositions.

Administration of pharmaceutical compositions according to the present invention will be via any common route so long as the target tissue is available via that route. Such routes include oral, nasal, buccal, rectal, vaginal or topical route. Alternatively, administration may be by intradermal, subcutaneous, intramuscular, intratumoral, intraperitoneal, or intravenous injection. Administration local or region to as tumor, or into the tumor vasculature, also are contemplated. Such compositions would normally be administered as pharmaceutically acceptable compositions, described supra.

The active compounds May also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, particular methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and anti fungal agents, isotonic and absorption delaying agents and the like. The use of such media and agents for pharmaceutical active substances is well known in the art, Except insofar as any conventional media or agent is incompatible with the active ingredient, its use in the therapeutic compositions is contemplated. Supplementary active ingredients can also be incorporated, into the compositions.

For oral administration the ligand traps or vectors coding therefor may be incorporated with excipients and used in the form of non-ingestible mouthwashes and dentifrices. A mouthwash may be prepared incorporating the active ingredient in the required amount in an appropriate solvent, such us a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in as therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage harms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The molecules according to the present invention can be delivered by encapsulating or embedding in a delivery vehicle. For example, liposomes, which are artificially prepared vesicles made of lipid bilayers have been used to delivery a variety of drugs. Liposomes can be composed of naturally-derived phospholipids with mixed lipid chains (like egg phosphatidylethanolamine) or other surfactants. In particular, liposomes containing cationic or neutral lipids have been used in the formulation of drugs. Liposomes should not be confused with micelles and reverse micelles composed of monolayers, which also can be used for delivery.

Nanoparticles are generally considered to be particulate substances having a diameter of 100 nm or less. In contrast to liposomes, which are hollow, nanoparticles tend to be solid. Thus, the drug will be less entrapped and more either embedded in or coated on the nanoparticle. Nanoparticles can be made of metals including oxides, silica, polymers such as polmethyl methacrylate, and ceramics. Similarly, nanoshells arc somewhat larger and encase the delivered substances with these same materials. Either nanoparticles or nanoshells permit sustained or controlled release of the peptide or mimetic, and can stabilize it to the effects of in vim environment.

Another modification for delivery of polypeptides is PEG-ylation. PEG-ylation is the process of covalent attachment of polyethylene glycol polymer chains to another molecule, normally a drug or therapeutic protein. PEG-ylation is routinely achieved by incubation of a reactive derivative of PEG with the target macromolecule. The covalent attachment of PEG to a drug, or therapeutic protein can “mask” the agent from the hosts immune system (reduced immunogenicity and antigenicity), increase the hydrodynamic size (size in solution) of the agent which prolongs its circulatory time by reducing renal clearance. PEG-ylation can also provide water solubility to hydrophobic drugs and proteins. Exemplary PEG-ylation technologies are described in U.S. Pat. Nos. 7,666,400, 7,610,156, 7,587,286, 6,552,170 and 6,420,339.

B. Inflammatory Disease States and Conditions

1. Cancer

Cancer results from the outgrowth of a clonal population of cells from tissue. The development of cancer, referred to as carcinogenesis, can be modeled and characterized in a number of ways. All association between the development of cancer and inflammation has long-been appreciated. The inflammatory response is involved in the host defense against microbial infection, and also drives tissue repair and regeneration. Considerable evidence points to a connection between inflammation and a risk of developing cancer, i.e., chronic inflammation can lead to dysplasia.

Studies have estimated that nearly 15% of worldwide cancer is associated with microbial infection. Organisms such as human papilloma virus (HPV), hepatitis B and C virus, HIV, and Helicobacter pylori all have been linked to cancer. In other cases, environmental conditions causing chronic irritation and subsequent inflammation can also predispose to cancer, including cigarette smoke, asbestos and silica.

In the case of some types of viral infection, virally-encoded genes can contribute to cellular transformation. An example is the HPV oncoproteins E6 and E7. However, other microbes associated with cancer do not operate in this fashion as they are not transforming. For example, certain strains of H pylori contain factors that ailed host cell signaling but do not contain oncogenes. Interestingly, it has been observed that H. pylori induces MUC1.

Other ways in which chronic inflammatory states can lead to genomic lesions and tumor initiation are chemical. For example, host cells fight microbial infection by the production of free radicals. In addition to their anti-microbial effects, these molecules lead to oxidative damage and nitration of DNA bases which increases the risk of DNA mutations even in host cells.

Yet another path to cellular dysregulation may result from the cell death that occurs in infection or other inflammatory insult. Lost cells must be repopulated by the expansion of other cells, sometimes undifferentiated precursor cells such as tissue stem cells. Not surprisingly, many inflammatory pathways function to mediate survival and proliferation. Thus, in attempting to mediating tissue repair, the inflammatory response may unwittingly provide excessive survival and proliferative signals to cells, thus leading to tumorigenesis.

Because of the link between cancer and inflammation, the ability of the peptides and peptide analogs of the present invention to reduce inflammatory signalling pathways can be exploited in a pre-cancer or cancer risk situation to prevent or delay the onset of dysplastic growth. Cancer cells to which the methods of the present invention can be applied include generally any cell that expresses MUC1, and more particularly, that overexpresses MUC1. An appropriate cancer cell can be a breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, cervical cancer, vaginal cancer, or bladder cancer cell. In addition, the methods of the invention can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice. Cancers may also be recurrent, metastatic and/or multi-drug resistant, and the methods of the present invention may be particularly applied to such cancers so as to prolong or re-induce remission, to prevent or limit metastasis, and/or to treat multi-drug resistant cancers.

The inventors propose that the local or regional delivery of MUC1 peptides to patients with cancer will be a very efficient method for treating the clinical disease. Similarly, the chemo- or radiotherapy (in combinations, as discussed below) may be directed to a particular, affected region of the subject's body. Alternatively, regional or systemic delivery of the agent(s) may be appropriate in certain circumstances, for example, where metastasis has occurred.

2. Sepsis

Sepsis is a serious medical condition characterized by a whole-body inflammatory state caused by infection. Traditionally the term sepsis has been used interchangeably with septicaemia and septicemia (“blood poisoning”). However, these terms are no longer considered synonymous; septicemia is considered a subset of sepsis.

Symptoms of sepsis are often related to the underlying infectious process. When the infection crosses into sepsis, the resulting symptoms are that of systemic inflammatory response syndrome (SIRS); general inflammation, fever, elevated white. blood cell count (leukocytosis), and raised heart rate (tachycardia) and breathing rate (tachypnea). Secondary to the above, symptoms also include flu like chills.

The immunological response that causes sepsis is a systemic inflammatory response causing widespread activation of inflammation and coagulation pathways. This may progress to dysfunction of the circulatory system and, even under optimal treatment. may result in the multiple organ dysfunction syndrome and eventually death.

Sepsis is considered present if infection is highly suspected or proven and two or more of the following systemic inflammatory response syndrome (SIRS) criteria are met:
heart rate>90 beats per minute
body temperature<36 (96.8° F.) or >38° C. (100.4° F.)
hyperventilation (high respiratory rate)>20 breaths per minute or, on blood gas, a PₐCO₂ less than 32 mm Hg,
white blood cell count<4000 cells/mm³ or >12000 cells/min³ (<4×10⁹ or >12×10⁹ cells/L), or greater than 10% hand forms (immature white blood cells).
   Consensus definitions however continue to evolve with the latest expanding the list of signs and symptoms of sepsis to reflect clinical bedside experience.

The more critical subsets of sepsis are severe sepsis (sepsis with acute organ dysfunction) and septic shock (sepsis with refractory arterial hypotension). Alternatively, when two or more of the systemic inflammatory response syndrome criteria are met without evidence of infection, patients may be diagnosed simply with “SIRS,” Patients with SIRS and acute organ dysfunction may be termed “severe SIRS.”

Patients are defined as having “severe sepsis” if they have sepsis plus signs of systemic hypoperfusion; either end organ dysfunction or a serum lactate greater than 4 mmol/dL. Patient are defined as having septic shock if they have sepsis plus hypotension after an appropriate fluid bolus (typically 20 ml/kg of crystaloid). The criteria for diagnosing an adult with sepsis do not apply to infants under one month of age. In infants, only the presence of infection plus a “constellation” of signs and symptoms consistent with the systemic response to infection are required for diagnosis.

The therapy of sepsis rests on antibiotics, surgical drainage of infected fluid collections, fluid replacement and appropriate support for organ dysfunction. This may include hemodialysis in kidney failure, mechanical ventilation in pulmonary dysfunction, transfusion of blood products, and drug and fluid therapy for circulatory failure. Ensuring adequate nutrition, if necessary by parenteral nutrition, is important during prolonged illness.

A problem in the adequate management of septic patients has been the delay in administering therapy after sepsis has been recognized, Published studies have demonstrated that for every hour delay in the administration of appropriate antibiotic therapy there is an associated 7% rise in mortality. A large international collaboration was established to educate people about sepsis and to improve patient outcomes with sepsis, entitled the “Surviving Sepsis Campaign.” The Campaign has published an evidence-based review of management strategies for severe sepsis, with the aim to publish a complete set of guidelines in subsequent years.

Most therapies aimed at the inflammatory process itself have failed to improve outcome, however drotrecogin alfa (activated protein C, one of the coagulation factors) has been shown to decrease mortality from about 31% to about 25% in severe sepsis. To qualify for drotrecogin alfa, a patient must have severe sepsis or septic shock with an APACHE II score of 25 or greater and a low risk of Weeding. Low dose hydrocortisone treatment has shown promise for septic shock patients with relative adrenal insufficiency as defined by ACTH stimulation testing.

Standard treatment of infants with suspected sepsis consists of supportive care, maintaining fluid status with intravenous fluids, and the combination of a β-lactam antibiotic (such as ampicillin) with an aminoglycoside such as gentamicin.

3. Trauma

Physical trauma is a serious and body-altering physical injury, such as the removal of a limb. Blunt force trauma, a type of physical trauma caused by impact or other force applied from or with a blunt object, whereas penetrating trauma is a type of physical trauma in which the skin or tissues are pierced by an object. Trauma can also be described as boat unplanned, such as an accident, or planned, in the case of surgery. Both can be characterized by mild to severe tissue damage, blood loss and/or shock, and both may lead to subsequent infection, including sepsis. The present invention provides to treatment of trauma, including, both pre-treatment (in the case of a medical procedure) and treatment after trauma injury as occurred.

Surgery.

Surgery uses operative manual and instrumental techniques on a patient to investigate and/or treat a pathological condition such as disease or injury, to help improve bodily function or appearance, or sometimes for some other reason. The present invention can address trauma resulting from surgeries, as defined further below.

As a general rule, a procedure is considered surgical when it involves cutting of a patient's tissues or closure of a previously sustained wound. Other procedures that do not necessarily fall under this rubric, such as angioplasty or endoscopy, may be considered surgery if they involve common surgical procedure or settings, such as use of a sterile environment, anesthesia, antiseptic conditions, typical surgical instruments, and suturing or stapling. All forms of surgery are considered invasive procedures; so-called noninvasive surgery usually refers to an excision that does not penetrate the structure being addressed (e.g., laser ablation of the cornea) or to a radiosurgical procedure (e.g., irradiation of a tumor). Surgery can last from minutes to hours.

Surgical procedures are commonly categorized by urgency, type of procedure, body system involved, degree of invasiveness, and special instrumentation. Elective surgery is done to correct a non-life-threatening condition, and is carried out at the patient's request, subject to the surgeon's and the surgical facility's availability. Emergency surgery is surgery which must be done quickly to save life, limb, or functional capacity. Exploratory surgery is performed to aid or confirm a diagnosis. Therapeutic surgery treats a previously diagnosed condition.

Amputation involves cutting off a body part, usually a limb or digit. Replantation involves reattaching a severed body part. Reconstructive surgery involves reconstruction of an injured, mutilated, or deformed part of the body. Cosmetic surgery is done to improve the appearance of an otherwise normal structure. Excision is the cutting out of an organ, tissue, or other body part from the patent. Transplant surgery is the replacement of an organ or body part by insertion of another from different human (or animal) into the patient. Removing an organ or body part from a live human or animal for use in transplant is also a type of surgery.

When surgery is performed on one organ system or structure, it may be classed by the organ, organ system or tissue involved. Examples include cardiac surgery (performed on the heart), gastrointestinal surgery (performed within the digestive tract and its accessory organs), and orthopedic surgery (performed on bones and/or muscles).

Minimally invasive surgery involves smaller outer incision(s) to insert miniaturized instruments within a body cavity or structure, as in laparoseopic surgery or angioplasty. By contrast, an open surgical procedure requires a large incision to access the area of interest. Laser surgery involves use of a laser for cutting tissue instead of a scalpel or similar surgical instruments. Microsurgery involves the use of an operating microscope for the surgeon to sec small structures. Robotic surgery makes use of a surgical robot, such as Da Vinci or Zeus surgical systems, to control the instrumentation under the direction of the surgeon.

Traumatic Hemorrhage.

Traumatic hemorrhage accounts for much of the wide ranging international impact of injury, causing a large proportion of deaths and creating great morbidity in the injured. Despite differences in pre-hospital care, the acute management of traumatic hemorrhage is similar around the world and follows well accepted published guidelines. A critically injured patient's care occurs as four, often overlapping segments: the resuscitative, operative, and critical care phases. The diagnosis and control of bleeding should be a high priority during all of the phases of trauma care and is especially important in the patient who is in hemorrhagic shock. Early attempts at hemorrhage control include direct control of visible sources of severe bleeding with direct pressure, pressure dressings, or tourniquets; stabilization of long bone and pelvic fractures; and keeping the patient warm. During the resuscitative phase, warmed intravenous fluids, hypotensive resuscitation prior to surgical control of hemorrhage, and appropriate transfusion of blood and blood products are provided. In the operative phase, surgical control of the hemorrhage and any other injury, and additional transfusion is provide, Finally, the critical care phase provides for post-operative support and tissue perfusion.

4. Acute Pancreatitis

Acute pancreatitis is rapidly-onset inflammation of the pancreas. Depending on its severity, it can have severe complications and high mortality despite treatment. While mild cases are often successfully treated with conservative measures or laparoscopy, severe cases require invasive surgery (often more than one intervention) to contain the disease process.

5. Acute Respiratory Distress Syndrome

Acute respiratory distress syndrome (ARDS), also known as respiratory distress syndrome (RDS) or adult respiratory distress syndrome (in contrast with IRDS) is a serious reaction to various forms of injuries to the lung. This is the most important disorder resulting in increased permeability pulmonary edema.

ARDS is a severe lung disease caused by a variety of direct and indirect insults. It is characterized by inflammation of the lung parenchyma leading to impaired gas exchange with concomitant systemic release of inflammatory mediators causing inflammation, hypoxemia and frequently resulting in multiple organ failure. This condition is life threatening and often lethal, usually requiring mechanical ventilation and admission to an intensive care unit. A less severe form is called acute lung injury (ALI).

ARDS can occur within 24 to 4 hours of an injury or attack of acute illness. In such a case the patient usually presents with shortness of breath, tactypnea and symptoms related to the underlying cause, i.e., shock. Long term illnesses can also trigger it, such as malaria. The ARDS may then occur sometime after the onset of a particularly acute case of the infection.

An arterial blood gas analysis and chest X-ray allow formal diagnosis by inference using the aforementioned criteria. Although severe hypoxemia is generally included, the appropriate threshold defining abnormal Path has never been systematically studied. Any cardiogenic cause of pulmonary edema should be excluded. This can be done by placing a pulmonary artery catheter for measuring the pulmonary artery wedge pressure. However, this is not necessary and is now rarely done as abundant evidence has emerged demonstrating that the use of pulmonary artery catheters does not lead to improved patient outcomes in critical illness including ARDS. Plain Chest X-rays are sufficient to document bilateral alveolar infiltrates in the majority of cases. While CT scanning leads to more accurate images of the pulmonary parenchyma in ARDS, its has little utility in the clinical management of patients with ARDS, and remains largely a research tool.

Acute respiratory distress syndrome is usually treated with mechanical ventilation in the Intensive Care Unit. Ventilation is usually delivered through oro-tracheal intubation, or tracheostomy whenever prolonged ventilation (≧2 weeks) is deemed inevitable. The possibilities of non-invasive ventilation are limited, to the very early period of the disease or, better, to prevention in individuals at risk for the development of the disease (atypical pneumonias, pulmonary contusion, major surgery patients). Treatment of the underlying cause is imperative, as it tends to maintain the ARDS picture. Appropriate antibiotic therapy must be administered as soon as microbiological culture results are available. Empirical therapy may be appropriate if local microbiological surveillance is efficient. More than 60% ARDS patients experience a (nosocomial) pulmonary infection either before or after the onset of lung injury. The origin of infection, when surgically treatable, must be operated on. When sepsis is diagnosed, appropriate local protocols should be enacted.

6. Ischemia-Reperfusion Injury

Reperfusion injury refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function.

The damage of reperfusion injury is due in part to the inflammatory response of damaged tissues. White blood cells carried to the area by the newly returning blood release a host of inflammatory factors such as interleukins a well as free radicals in response to tissue damage. The restored blood flow reintroduces oxygen within, cells that damages cellular proteins, DNA, and the plasma membrane. Damage to the cell's membrane may in turn cause the release of more free radicals. Such reactive species may also act indirectly in redox signaling to turn on apoptosis. Leukocytes may also build of in small capillaries, obstructing them and leading to more ischemia.

Reperfusion injury plays a part in the brain's ischemic cascade, which is involved in stroke and brain trauma. Repeated bouts of ischemia and reperfusion injury also arc thought to be a factor leading to the formation and failure to heal of chronic wounds such as pressure sores and diabetic foot ulcers. Continuous pressure limits blood supply and causes ischemia, and the inflammation occurs during reperfusion. As this process is repeated, it eventually damages tissue enough to cause a wound.

In prolonged ischemia (60 min or more), hypoxanthine is formed as breakdown product of ATP metabolism. The enzyme xanthine dehydrogenase is converted to xanthine oxidase as a result of the higher availability of oxygen. This oxidation results in molecular oxygen being converted into highly reactive superoxide and hydroxyl radicals, Xanthine oxidase also produces uric acid, which may act as both a prooxidant and as a scavenger of reactive species such as peroxinitrite. Excessive nitric oxide produced during reperfusion reacts with superoxide to produce the potent reactive species peroxynitrite. Such radicals and reactive oxygen species attack cell membrane lipids, proteins, and glycosaminoglycans, causing further damage. They may also initiate specific biological processes by redox signaling.

7. Cardiovascular Disease

Cardiovascular disease refers to the class of diseases that involve the heart or blood vessels (arteries and veins). While the term technically refers to any disease that affects the cardiovascular system, it is usually used to refer to those related to atherosclerosis (arterial disease). These conditions have similar causes, mechanisms, and treatments. Treatment of cardiovascular disease depends on the specific form of the disease in each patient, but effective treatment always includes preventive lifestyle changes discussed above. Medications, such as blood pressure reducing medications, aspirin and the statin cholesterol-lowering drugs may be helpful. In some circumstances, surgery or angioplasty may be warranted to reopen, repair, or replace damaged blood vessels

Most Western countries face high and increasing rates of cardiovascular disease. Each year, heart disease kills more Americans than cancer. Diseases of the heart alone caused 30% of all deaths, with other diseases of the cardiovascular system causing substantial further death and disability. Up until the year 2005, it was the number 1 cause of death and disability in the United States and most European countries. A large histological study (PDAY) showed vascular injury accumulates from adolescence, making primary prevention efforts necessary from childhood.

Some biomarkers arc thought to offer a more detailed risk of cardiovascular disease. However, the clinical value of these biomarkers is questionable. Currently, biomarkers Which may reflect a higher risk of cardiovascular disease include:
higher fibrinogen and PAI-1 blood concentrations
hlevated homocysteine, or even upper half of normal
elevated blood levels of asymmetric dimethylarginine
high inflammation as measured by C-reactive protein
levated blood levels of B-type natriuretic peptide (BNP)
   Various forms of cardiovascular disease include aneurysms, angina, arrhythmia, atherosclerosis, cardiomyopathy, cerebrovascular disease, congenital heart disease, congestive heart failure, myocarditis, valve disease, coronary artery disease, dilated cardiomyopathy, diastolic dysfunction, endocarditis, high blood pressure (hypertension), hypertrophic cardiomyopathy, nitral valve prolapse, myocardial infarction, and venous thromboembolism.

8. Autoimmune/Inflammatory Disease

The present invention contemplates the treatment of a variety of autoimmune and/or inflammatory disease states such as spondyloarthropathy, ankylosing spondyiitis, psoriatic arthritis, reactive arthritis, enteropathic arthritis, ulcerative colitis, Crohn's disease, irritable bowel disease, inflammatory bowel disease, rheumatoid arthritis, juvenile rheumatoid arthritis, familial Mediterranean fever, amyotrophic lateral sclerosis, Sjogren's syndrome, early arthritis, viral arthritis, multiple sclerosis, or psoriasis. The diagnosis and treatment of these diseases are well documented in the literature.

9. Chemotherapy, Radiotherapy and Cytokine Therapy Toxicity

Various forms of cancer therapy, including chemotherapy, radiation, and cytokines, are associated with toxicity, sometimes severe, in the cancer patient. To the extent that the toxicity is caused at least in part by the ex tracellular actions of histories, the present invention seeks to reduce this toxicity using the pharmaceutical compositions of the present invention, thereby reducing or alleviating discomfort on the part of the patient, as well as permitting higher doses of the therapy.

10. Burns

In medicine, a burn may be an injury caused by heat, cold, electricity, chemicals, friction or radiation. First-degree burns are usually limited to redness (erythema), a white plaque, and minor pain at the site of injury. These burns usually extend only into the epidermis. Second-degree burns additionally fill with clear fluid, have superficial blistering of the skin, and can involve more or less pain depending on the level of nerve involvement. Second-degree burns involve the superficial (papillary) dermis and may also involve the deep (reticular) dermis layer. Third-degree burns additionally have charring of the skin, and produce hard, leather-like eschars. An eschar is a scab that has separated from the unaffected part of the body. Frequently, there is also purple fluid. These types of burns are often painless, because nerve endings have been destroyed in the burned areas. Serious burns, especially if they cover large areas of the body, can cause death; any hint of burn injury to the lungs (e.g., through smoke inhalation) is a medical emergency.

Burns that injure the tissues underlying the skin, such as the muscles or bones, are sometimes categorized as fourth-degree burns. These burns are broken down into three additional degrees: fourth-degree burns result in the skin being irretrievably lost, fifth-degree burns result in muscle being irretrievably lost, and sixth-degree burns result in bone being charred.

A newer classification of “Superficial Thickness,” “Partial Thickness” (which is divided into superficial and deep categories) and “Full Thickness” relates more precisely to the epidermis, dermis and subcutaneous layers of skin and is used to guide treatment and predict outcome.

Chemical burns are usually caused by chemical compounds, such as sodium hydroxide (lye), silver nitrate, and more serious compounds (such as sulfuric acid). Most chemicals (but not all) that can cause moderate to severe chemical burns are strong acids or bases. Nitric acid, as an oxidizer, is possibly one of the worst burn-causing chemicals. Hydrofluoric acid can eat down to the bone and its burns are often not immediately evident. Most chemicals that can cause moderate to severe chemical burns are called caustic.

Electrical burns are generally symptoms of electric shock, being struck by lightning, being defibrillated or cardioverted without conductive gel, etc. The internal injuries sustained may be disproportionate to the size of the “burns” seen—as these are only the entry and exit wounds of the electrical current.

Burns are assessed in terms of total body surface area (TBSA), which is the percentage affected by partial thickness or full thickness burns (superficial thickness hums are not counted). The rule of nines is used as a quick and useful way to estimate the affected TBSA. The first step in managing a person with a burn is to stop the burning process. With dry powder burns, the powder should be brushed off first. With other burns, the affected area should be rinsed with a large amount of clean water to remove foreign bodies and help stop the burning process. Cold water should never be applied to any person with extensive burns, as it may severely compromise the burn victim's temperature status. At this stage of management, it is also critical to assess the airway status. If the patient was involved in a tire, then it must be assumed that he or she has sustained inhalation injury until proven otherwise, and treatment should be managed accordingly.

Once the burning process has been stopped, and airway status is ensured, the patient should be volume resuscitated according to the Parkland formula. This formula dictates that the amount of Lactated Ringer's solution to deliver in the first twenty four hours after time of injury is:

fluid 4 cc×% TBSA×weight in kg
% TBSA excludes any first degree burn

Half of this fluid should be given in the first eight hours post injury and the rest in the subsequent sixteen hours. The formula is a guide only and infusions must be tailored to urine output and central venous pressure. Inadequate fluid resuscitation causes renal failure and death. Severe edema in full thickness burns may be treated by escharotomy.

C. Treatment Methods

Ligand traps or vectors coding therefor can be administered to mammalian subjects (e.g., human patients) alone or in conjunction with other drugs that modulate disease states. The compounds can also be administered to subjects that are genetically and/or duo to, for example, physiological and/or environmental factors, susceptible to disease states, e.g., subjects with a family history of disease, subjects with chronic disease, and in particular subjects having or at risk of cancer.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.0001-100 mg/kg. Wide variations in the needed dosage are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g. 2-, 3-, 4-, 6-, 8-, 10-, 20-, 50-, 100-, 150-, or more times). Encapsulation of the polypeptide/vector in a suitable delivery vehicle (e.g., polymeric microparticles or implantable devices) may increase the efficiency of delivery, particularly for oral delivery.

D. Combination Therapies

It is common in many fields of medicine to treat a disease with multiple therapeutic modalities, often called “combination therapies.” Inflammatory disorders, such as cancer, are no exception.

To treat cancers using the methods and compositions of the present invention, one would generally contact a target cell or subject with a MUC1 ligand trap and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the MUC1 ligand trap and the other includes the other agent.

Alternatively, the MUC1 ligand trap may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the MUC1 antagonist or the other therapy will be desired. Various combinations may be employed, where the MUC1 ligand trap or vector coding therefor) is “A,” and the other therapy is “B” as exemplified below;
A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B
   Other combinations are contemplated. To kill cells, inhibit cell growth, inhibit metastasis, inhibit angiogenesis or otherwise reverse or reduce the malignant phenotype of tumor cells, using the methods and compositions of the present invention, one may contact a target cell with a MUC1 ligand trap and at least one other therapy. These therapies would be provided in a combined amount effective to kill or inhibit proliferation of the cell. This process may involve contacting the cells with the agents/therapies at the same time. This may be achieved by contacting the cell with a single composition or pharmacological formulation. that includes both agents, or by contacting the cell with two distinct compositions or formulations, at the same time, wherein, one composition includes the MUC1 ligand trap and the other includes the agent.

Agents or factors suitable for use in a combined therapy include any chemical compound or treatment method that induces DNA damage when applied to a cell. Such agents and factors include radiation and waves that induce DNA damage such as, irradiation, microwaves, electronic emissions, and the like. A variety of chemical compounds, also described as “chemotherapeutic” or “genotoxic agents” are intended to be of use in the combined treatment methods disclosed herein. In treating cancer according to the invention, one would contact the tumor cells with an agent in addition to the expression construct. This may be achieved by irradiating the localized tumor site; alternatively, the tumor cells may be contacted with the agent by administering to the subject a therapeutically effective amount of a pharmaceutical composition.

Various classes of chemotherapeutic agents are comtemplated for use with in combination with peptides of the present invention. For example, selective estrogen receptor antagonists (“SERMs”), such as Tamoxifen, 4-hydroxy Tamoxifen (Afimoxfene), Falsodex, Raloxifenc, Bazedoxifene, Clomifene, Femarelle, Lasofoxifene, Ormeloxifene, and Toremifene.

Chemotherapeutic agents contemplated to be of use, include, e.g., camptothecin, actinomycin-D, mitomycin C. The invention also encompasses the use of a combination of one or more DNA damaging agents, whether radiation-based or actual compounds, such as the use of X-rays with cisplatin or the use of cisplatin with etoposide. The agent may be prepared and used as a combined therapeutic composition, or kit, by combining it with a MUC1 peptide, as described above.

Heat shock protein 90 is a regulatory: protein found in many eukaryotic cells HSP90 inhibitors have been shown to be useful in the treatment of cancer. Such inhibitors include Geldanamycin, 17-(Allylamino)-17-demethoxygeldanamycin PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxoruhicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for doxorubicin, to 35-50 mg/m² for etoposide intravenously or double the intravenous dose orally. Microtubule inhibitors, such as taxanes, also are contemplated. These molecules are diterpenes produced by the plants of the genus Taxus, and include paclitaxel and docetaxel.

Epidermal growth factor receptor inhibitors, such as Iressa, mTOR, the mammalian target of rapamycin, also known as FK506-binding protein 12-rapamycin associated protein 1 (FRAP1) is a serine/threonine protein kinase that regulates cell growth, cell proliferation, cell motility, cell survival, protein synthesis, and transcription. Rapamycin and analogs thereof (“rapalogs”) are therefore contemplated for use in combination cancer therapy in accordance with the present invention.

Another possible combination therapy with the peptides claimed herein is TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenosis and viral replication.

Agents that disrupt the synthesis and fidelity of nucleic acid precursors and subunits also lead to DNA damage. As such as number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU. is applicable in a wide range of carriers, including topical, however intravenous administration with doses ranging from 3 to 15 mg/kg/day being commonly used.

Other factors that cause DNA damage and have been used extensively include what are commonly known as γ-rays, x-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In addition to combining MUC1 ligand trap therapies with chemo- and radiotherapies, it also is contemplated that combination with immunotherapy, hormone therapy, toxin therapy and surgery. In particular, one may employ targeted therapies such as Avastin, Erbitux, Cleaver, Herceptin and Rituxan.

The present invention also contemplates particular combinations wherein the “other agent” also targets MUC1, but in a different manner than the ligand trap. In particular, this contemplates the use of oligomer inhibiting peptides, such as those described in U.S. Patent Publications 2011/0015138 and 2010/012505 and Ser. No. 13/026,858, and small molecules as described in U.S. Ser. No. 13/045,033, each of which are incorporated by reference.

It also should be pointed out that any of the foregoing therapies may prove useful by themselves in treating cancer.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### In Vitro Studies

Generation of MUC1-C-ED-Fc Chimeric Proteins—Glycosylated (GO-101) and Unglycosylated (GO-107).

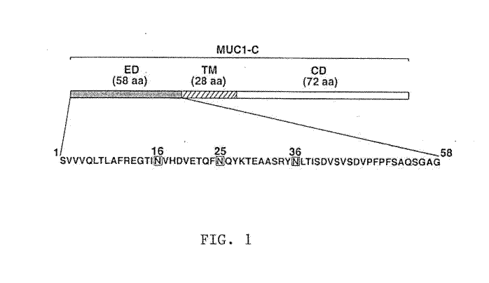

Human MUC1-C-ED (FIG. 1) was fused with the PCR-amplified mouse or human IgG Fc fragment and cloned into the pCR3.1 vector between NheI and XbaI sites. The pCR3.1 in Fc-MUC1-C-ED or pCR3.1 hFc-MUC1-C-ED plasmids were stably transfected into the Chinese Hamster Ovarian (CHO) cell line CHO-K1. Separately, these plasmids were also transfected into mutant Lec1-CHO which lack GlcNAc glycosyl transferase so that N-linked carbohydrates are blocked at the Man5-GlcNAC2-Asn intermediate. This cell line is used to produce the unglycosylated in Fc-MUC1-C-ED and hFc-MUC1-C-ED chimeric proteins. These stable cell lines were grown in BD cell monoclonal antibody production chambers (Cell line 1000) in serum free media. Secreted proteins in the supernatant were passed through protein-A column, washed extensively and eluted with 0.1 M Citric acid, pH 3.0. The eluted proteins were concentrated and run on SDS-PAGE gels to confirm size and purity. The human Fc-MUC1-C-ED glycosylated protein is designated as GO-101 and unglycosylated protein, is designated as GO-102.

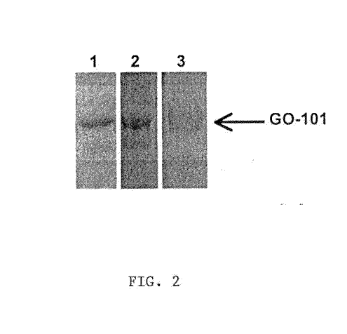

Based on the data from initial in vitro cell proliferation experiments, further in-depth experiments were performed with GO-101. The production of GO-101 protein using CHO-K1 stably transfected with pCR3.1 hFc-MUC1-C-ED vector is very low (˜100 μg/L). Hence, multiple approaches were engaged in order to obtain sufficient amount of GO-101 protein for various in vitro experiments: i) outsourcing the production to XTAL BioStructures Inc. for producing 5-6 mgs of protein (FIG. 2) and ii) generation of new mammalian expression vectors with a linker between hFc and MUC1-C-ED, Mid-scale GO-101 protein production (˜25 mg) for initial ZR-75-1 breast carcinoma xenograft study was contracted out to HyproCell Inc, in Connecticut (FIG. 2).
Xtal BioStructures Inc., Natick, Mass. Five lugs of hFc-MUC1-C-ED protein was generated and purified for multiple in vitro assays,
HyproCell Inc., Connecticut. Twenty mgs of hFc-MUC1-C-ED protein was generated and purified for in vivo human tumor mice xenograft study.
   Representative gels from the production of different batches of GO-101 protein is shown in FIG. 2.

Inhibition of Estrogen-Dependent Human Breast Carcinoma Cell Proliferation by GO-101 In Vitro.

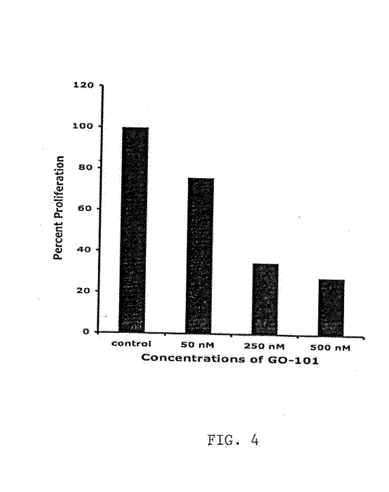

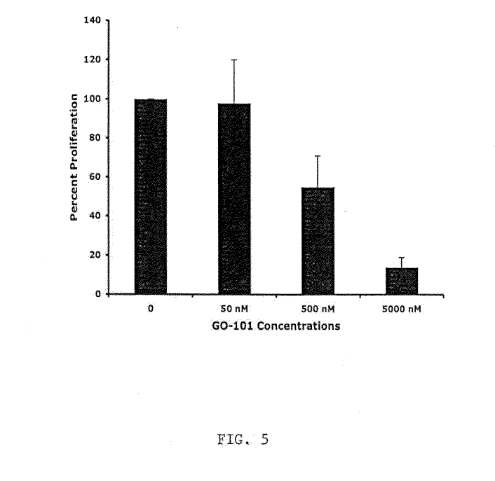

Human ZR-75-1 breast carcinoma cells (10,000 cells/well) were plated into 24-well tissue culture plates and grown for 24 h at 37° C. Cells were then independently treated each day with different concentrations (50 nM, 250 nM and 500 nM) of GO-101. All treatment points were set-up in triplicate. As a negative control, cells were also treated with same concentrations of purified hFc protein. Plates will be subsequently incubated for an additional 3 days. After the end of the incubation period, cell proliferation was determined using trypan blue exclusion method. The percent proliferation of ZR-75-1 cells is shown in FIG. 4. Similar results were obtained when another estrogen-dependent breast carcinoma cell line MCF-7 was treated with different concentrations of GO-101 (FIG. 5).

Inhibition of Non-Small Cell Lung Carcinoma Cell Proliferation by go-101 In Vitro.

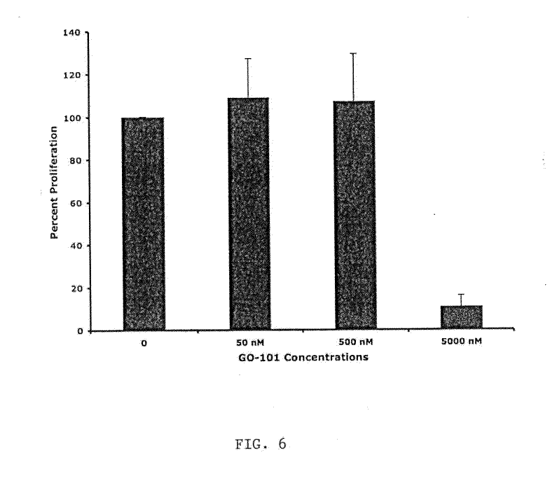

Human H 1975 non-small cell lung carcinoma cells (10,000 cells/well) were plated into 24-well tissue culture plates and grown for 24 h at 37e. Cells were then independently treated each day with different concentrations (50 nM, 500 nM and 5000 nM) of GO-101 for three days. All treatment points were set-up in triplicates. As a negative control, cells were also treated with same concentrations of purified hFc protein. After 3 days of treatment, cell proliferation was determined using trypan blue exclusion method. The percent proliferation of H1975 cells is shown in FIG. 6.

AlamarBlue Assay for Cell Proliferation/Cell Death.

The AlamarBlue assay involves the usage of a fluorometric/colorimetric growth indicator, AlamarBlue based on the Metabolic activity of cells. As cells grow, the innate metabolic activity results in a chemical reduction of AlamarBlue. Continued growth maintains a reduced environment while inhibition of growth maintains an oxidized environment. Live cells cause the change of AlamarBlue from oxidized (non-fluorescent, blue) form to reduced (fluorescent, red) form. Data can be collected using either fluorescence-based or absorbance-based instrumentation, Fluorescence is monitored at 530-560 nm excitation wavelength and at 590 nm emission wavelength. Absorbance is monitored at 570 nm and 600 nm. It is a quantitative method in which a known standard (drug) can be used and the effect of an unknown agent can be deduced by interpolation of the obtained values for the unknown against the standard curve.

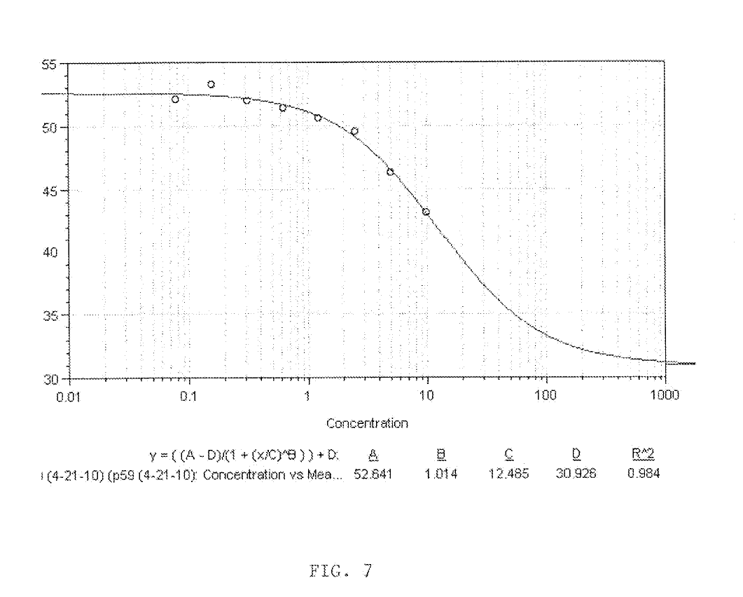

This assay was developed with a 96-well plate using H1650 (non-small cell lung carcinoma cell line) at different cell concentrations and the optimal cell number was decided to be 5,000 cells/well in 100 μl volume. The cell number/well has to be optimized for each cell type to be tested. Cells were treated with various concentrations of GO-101 starting from 10 μM with 2-fold serial dilutions to obtain a total of 8 different concentrations. Cells in duplicate wells were treated with each concentration of GO-101 every day, for 4 days. On day 5, the medium in the plate was replaced with 1.0% alamarBlue solution and incubated for various time points (1-5 hrs). At the end of each hour, absorbance of the plate was measured at 570 nm and 600 nm as reference. A set of blank wells were maintained with medium alone and medium with AlamarBlue for the purpose of calculating actual absorbance, Obtained absorbance values were plotted against respective concentrations of GO-101. FIG. 7 indicates a representative plot obtained throne of the assays performed with GO-101 as the killing agent using, H1650 cell line with the above mentioned conditions.

In this plot, A and D represent the values of the upper and lower asymptotes and 3 is the slope and C is the midpoint of the curve. C, being the mid point of the curve, can be used for deducing the LD₅₀ of the reagent under test. Accordingly, the data presented here shows the percent reduction of AlamarBlue is 52.85% at the lowest concentration tested (0.078 μM) which is close to the untreated cell control. Therefore, it can be considered as 100% viability for the cells at that level of AlamarBlue reduction. Analysis of the 4-parameter curve shows a dose-dependent decrease in the viable cells. The highest concentration tested was 10 μM. Due to lack of availability of purified p59-hFc, any further concentrations could not be tested that can yield LD value. En order to achieve that, further improvement of protein production and purification is required and efforts are underway as described.

Synergistic Inhibition of Cell Proliferation by GO-101 with Oxidative Stress.

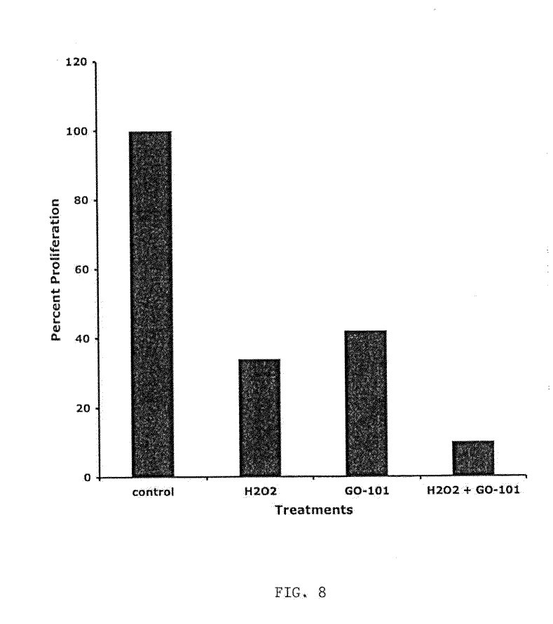

To assess the effects of GO-101 on sensitivity of human carcinoma cells to oxidative stress, the inventors treated ZR-75-1 breast carcinoma cells with 500 nM GO-101 in the presence and absence of 1.0 nM H2O2. As a control, cells were also separately treated with hFc (data not shown). Following treatment for 3 days, viable cells were then counted using trypan blue exclusion. The results demonstrate that ZR-75-1 cells exposed to H2O2 exhibited ˜66% inhibition in cell proliferation compared to mock conditions. Importantly, the ZR-75-1 cells exposed to 500 nM GO-101, but not hFc, was associated with ˜42% growth inhibition. However, there was a ˜90% growth inhibition when cells were treated with both H2O2 and GO-101 (FIG. 8).

Synergistic Inhibition of Cell Proliferation by GO-101 with Chemotherapeutic Agent, Doxorubicin.

ZR-75-1 human breast carcinoma cell were treated with various concentrations of GO-101 starting from 2 μM with 2-fold serial dilutions to obtain a total of 8 different concentrations in the presence or absence of doxorubicin starting from 25 nM with 2-fold serial dilutions to obtain a total of 8 different concentrations. Cells were also separately treated with multiple concentrations of doxorubicin starting from 25 nM with 2-fold serial dilutions to obtain a total of 8 different concentrations. Cells in duplicate wells were treated with either alone (doxorubicin or GO-101) or in combinations for 4 days. On day 5, the medium in the plate was replaced with 10% alamarBlue solution and incubated for various time points (1-5 hrs). At the end of each hour, absorbance of the plate was measured at 570 nm and 600 nm as reference. A set of blank wells were maintained with medium alone and medium with alamarBlue for the purpose of calculating actual absorbance. Obtained absorbance values were plotted against respective concentrations of GO-101, doxorubicin or in combination of GO-101 and doxorubicin

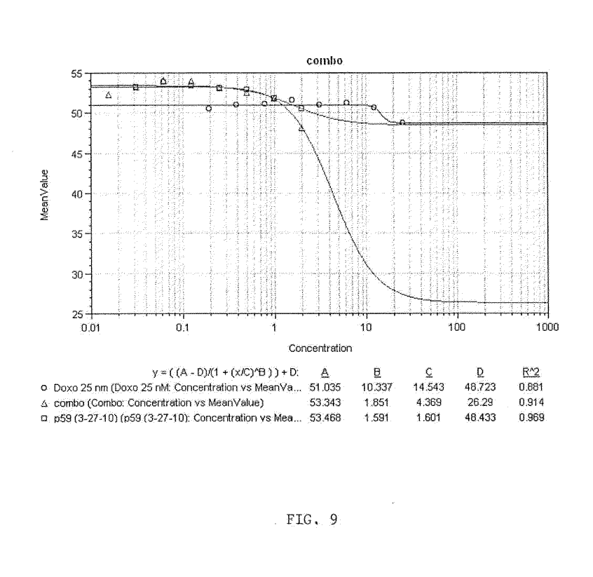

The results demonstrate that there was a very little inhibition of cell proliferation at low doses of either agent alone. However, there is a substantial reduction in cell proliferation when the two agents combined. FIG. 9 indicates a representative plot obtained for one of the assays performed with these conditions.

### Example 2

### In Vivo Dose Response Study of GO-101 in Hormone-Dependent ZR-75-1 Human Breast Carcinoma Tumor Xenograft Model

As mentioned above, the production of GO-101 protein using CHO-K1 stably transfected with pCR3.1 hFc-MUC1-C-ED vector is very low (˜100 μg/L). To study the effects of multiple doses of GO-101 on ZR-75-1 human breast carcinoma xenograft in mice required at least 75-100 mgs of protein. Hence, the inventors performed a pilot. study (2 doses only with 5 mice in each group) using a batch of 25 mgs of GO-101 produced and purified at HyproCell Inc. The study was performed at NIH-approved CRO, MIR/Charles River Labs Discovery and Imaging Services.

Animal Efficacy Study Protocol.

GO-101. To avoid freeze/thaw cycles, GO-101 protein (HC-batch GENU001) was prepared as multiple aliquots of frozen solution in PBS into individual treatment doses. The frozen tubes were stored at −80° C. until initiation of dosing. On each day of treatment, the appropriate tubes were allowed to thaw at room temperature. The resulting solution in each tube was clear at d colorless with a pH of ˜6.5.

Nu/Nu Mice.

About 9-10 weeks old female mice (on the day of treatment) (CRL: NUFoxn1nu) were obtained from Charles River Labs. The mice were fed irradiated Rodent Diet 5053 and water ad libitum. All the treatments, body weight determinations and tumor measurements were carried out in the bubble environment. Test animals were implanted subcutaneously, high in the right axilla, on Day 0 with 5×10⁶ cells/animal (0.2 ml) using a 27 gauge needle and syringe.

ZR-75-1 cells.

ZR-75-1 cells were obtained from ATCC and expanded using RPMI 1640 media supplemented with 10% Fetal Bovine Serum (heat inactivated), 1% penicillin-streptomycinglutamine, 1% HEPES, 1% Na-Pyruvate and 1% glucose in 5% CO2 atmosphere at 7° C. When expansion was complete, the cells were trypsinized and pooled for implantation. The ZR-75-1 (passage 7) cell, suspension was counted using trypan blue exclusion. A 2×10⁷ cells/ml suspension was prepared in scrum free RPMI 1640 with 50% Matrigel. The pre-injection viability was 95.8%

ZR-75-1 Human Breast Carcinoma.

ZR-75-1 human breast carcinoma is an estrogen-dependent tumor model, and all of the mice Were implanted with 17-β estradiol pellets (0.36 mg/pellet, 60-day release, Innovative Research of America). The pellets were implanted subcutaneously on the back of the neck with a 10-gauge trocar needle prior to tumor implant. Blood levels of 17-β estradiol were not monitored during the experiment.

Treatments.

Treatments began on Day 17, when the mean estimated tumor mass for all groups in the experiment was 136 mg (range of group means, 129-144). All animals weighed>23.8 g at the initiation of therapy. Mean group body weights at first treatment were well-matched (range 25.4-28 g). All animals dosed with either the vehicle or different concentrations of GO-101 were injected with a fixed volume of 100 d as shown in Table 5.

TABLE 5

| | | # of | | | Dose | |
|---|---|---|---|---|---|---|
| Group | Compound | Mice | Route | Schedule | (mg/kg) | |
| 1 | Vehicle | 5 | i.p. | QD × 21 | 100 | μl |
| 2 | GO-101 | 5 | i.p. | QD × 21 | 1 | mg/kg |
| 3 | GO-101 | 5 | i.p. | Q3D × 7 | 10 | mg/kg |

Measurements.

All mice were observed for clinical signs at least once daily. Mice with tumors in excess of ˜1 g or with ulcerated tumors were euthanized. All procedures were canned out in this experiment were conducted in compliance with all the laws, regulations and guidelines of the National Institutes of Health (NIH) and with the approval of Discovery and Imaging Services, Ann Arbor's (DIS-AA) Animal Care and Use Committee. DIS-AA is an AAALAC accredited facility. Body weights and tumor measurements were recorded twice weekly.

Tumor Burden.

Tumor burden (mg) was estimated from caliper measurements by the formula for the volume of a prolate ellipsoid assuming unit density as

tumor burden (mg)=(L×W2)/2

where L. and are the respective orthogonal tumor length and width measurements (mm).

Results.

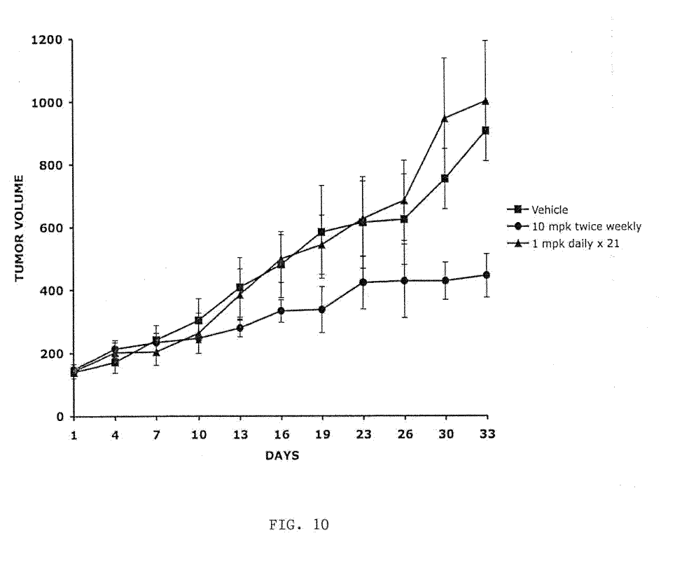

The mean estimated tumor mass for all groups on day 1 of treatment in the experiment was 136 mg (range of group means, 129-144). A tumor burden of ˜750 mg in the vehicle treated group was chosen for evaluation of any sign of efficacy by tumor growth delay. The median vehicle tumor reached evaluation size on Day 30. Treatment of GO-101 at 1 mg/kg daily×21 showed no sign of toxicity. Treatment with GO-101 twice weekly at 10 mg/kg was well tolerated, produced no treatment-related mortality and maximum treatment-related weight losses of ˜8%, Treatment of GO-101 at 1 mg/kg daily×21 failed to produce anti-cancer activity (p>0.05) (FIG. 10). However, treatment with GO-101 at 10 mg/kg twice weekly (only 6 doses for the entire study) produced some tumor growth inhibition starting at Day 16. These findings suggest a trend of increasing anti-cancer activity dependent on dose. Taken together, these results demonstrate that doses of GO-101 such as 5 mg/kg daily×21 or 10 mg/kg every other day×21 will be significantly effective in tumor growth inhibition.

Efforts arc underway to produce large quantities of GO-101 by using stable CHO-K1 cell line expressing the new hFc-MUC1-C-ED vector as described above. As discussed, due to the limitation of GO-101 protein, the inventors were unable to perform extensive dose-schedule efficacy studies with appropriate number of mice (10 mice) in each group. However, the results of the pilot efficacy study using human breast cancer xenograft model are very encouraging and warrants further evaluation with different doses and at multiple schedules.

### Example 3

### GO-101 and Doxorubicin Combination Study in Hormone-Dependent ZR-75-1 Human Breast Carcinoma Tumor Xenograft Model

As mentioned above, the production of GO-101 protein using CHO-K1 stably transfected with pCR3.1 hFc-MUC1-C-ED vector is very low. Hence, the inventors have performed GO-101 and doxorubicin combination study by using only one low dose of GO-101.

Study Protocol.

GO-101. To avoid freeze/thaw cycles, GO-101 protein (HG-batch GENU001) was prepared as multiple aliquots of frozen solution in PBS into individual treatment doses. The frozen tubes were stored at −80° C. until initiation of dosing. On each day of treatment, the appropriate tubes were allowed to thaw at room temperature. The resulting solution in each tube was clear and colorless with a pH of ˜6.5.

Doxorubicin.

Two mg/ml doxorubicin (lot #1705942, CRL/MIR) was manufactured by Bedford Labs and obtained from McKesson Speciality Care Solutions. Upon receipt, the clear, red solution was stored at 4° C. protected from light. Dosing solutions were prepared just prior to each treatment by dilution with saline.

Nu/nu Mice.

About 9-10 weeks old female mice (on the day of treatment) (CRL: NUFoxn1nu) were obtained from Charles River Labs. The mice were fed irradiated Rodent Diet 5053 and water ad libitum. All the treatments, body weight determinations and tumor measurements were carried out in the bubble environment. Test animals were implanted subcutaneously, high in the right axilla, on Day U with 5×106 cells/animal (0.2 using a 27 gauge needle and syringe, ZR-75-1 Cells were obtained from ATCC and expanded using RPMI 1640 media as described above. ZR-75-1 human breast carcinoma is an estrogen-dependent tumor model, and all of the mice were implanted with 17-β estradiol pellets (0.36 mg/pellet, 60 day release, Innovative Research of America). The pellets were implanted subcutaneously on the back of the neck with a 10-gauge trocar needle prior to tumor implant. Blood levels of 17-β estradiol were not monitored during the experiment. All animals dosed with either doxorubicin alone or in combination with 1 mg/kg GO-101 daily×2.1. Doxorubicin was injected 6 mg/kg i.v. Q4D×3 in 200 μl volume and GO-101 was injected with a fixed volume of 100 μl as shown in Table 6.

TABLE 6

| | | # of | | | Dose |
|---|---|---|---|---|---|
| Group | Compound | Mice | Route | Schedule | (mg/kg) |
| 1 | Doxorubicin | 5 | i.v. | Q4D × 3 | 6 mg/kg |
| 2 | GO-101 + | 5 | i.p./i.v. | QD × 21 | 1 mpk + |
| | Dox | | | | 6 mpk |

Results.

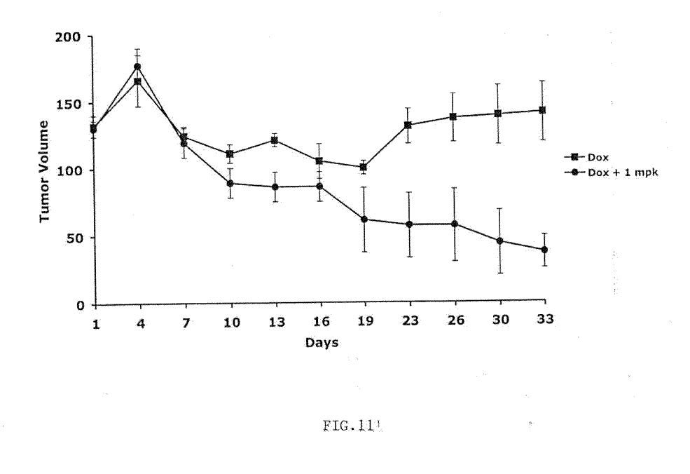

Treatment with doxorubicin at 6 mg/kg given every fourth day for three injections plus GO-101 (1 mg/kg, daily×21) was apparently tolerated, producing no “treatment-related” toxicity. Treatment with doxorubicin produced significant (p=0.0356) anti-tumor activity based upon tumor growth delay. Doxorubicin treatment produced neither tumor regressions nor tumor free survivors. Combination treatment with doxoruhicin+GO-101 produced a significant inhibition of tumor growth to that compared with Doxorubicin alone (FIG. 11). Two out of 5 animals in this group had complete tumor regressions. Although this treatment regimen appeared to produce more tumor regressions compared to single agent therapy, suggesting enhanced anti-cancer activity. Of note, the dose of GO-101 (1 mg/kg) had absolutely no anticancer activity stand alone (FIG. 10), doxorubicin-sensitization at this low dose level is very encouraging. Additional studies are planned to determine doxo- or other chemo-sensitization with 5 mg/kg GO-101 daily×21 days.

### Example 4

Methods.

Mice. Female mice (CRL, Wilmington, Mass.; BALB/c no/au) were obtained from Charles River Laboratories. They were 6-7 weeks old on Day 0 of the experiment at the time of tumor implantation. The mice were fed irradiated Rodent Diet 5053 (LabDiet™) and water ad libitum. The mice were housed in cages in Clean Rooms that provide H.E.P.A. filtered air into the bubble environment at 100 complete air changes per hour. All treatments, body weight determinations and tumor measurements were carried out in the bubble environment. The environment was controlled to a temperature range of 70°±2° F. and a humidity range of 30-70%.

Breast Carcinoma Cells and Tumor Implantation.

Human ZR-75-1 breast carcinoma cells were grown in RPMI 1640 supplemented with 10% heat-inactivated fetal bovine scrum, 100 units/ml penicillin and 100 mg/ml streptomycin. BALB/c nu/nu female mice were implanted subcutaneously (SC) with 17β-estradiol plugs (0.72 mg; Innovative Research) using a trocar gun. After 24 hours, 10 million ZR-75-1 cells were injected SC in the flank. Four to five of such animals were chosen as donors. Following the growth of ZR-75-1 cells in donor mice, the tumors were surgically and aseptically removed and transferred to a sterile Petri dish containing RPMI 1640 media (Bio-Whittaker). With the help of a hand held homogenizer, the tumors were homogenized by gently pushing the piston forward and backwards. The homogenate was centrifuged in a refrigerated centrifuge at 2,000 rpm for five minutes and a pellet was formed at the bottom of the test-tube. The pellet was washed three times by centrifuging and re-suspending the cells in the plain media without serum and last time with sterile PBS (pH 7.4). The cells were counted by using a hemocytometer and cell viability was determined by trypan blue elimination technique. A suspension of 20 million cells per milliliter of media was mixed with an equal volume of Matrigel (B&D Laboratories) to achieve a final concentration of 10 million cells in one mL of media. The mice were randomly selected into 2 groups of 4 mice. Using a tuberculin syringe and a 25G needle, 500 micro liters of the suspension was introduced in to the right thigh, taking precautions to prevent sample flush back. The animals were numbered by ear-punching. ZR-75-1, human breast carcinoma, is an estrogen-dependent tumor model and all of the mice were implanted with 17β-estradiol pellets. Blood levels of 17β-estradiol were not monitored during the experiment. All mice were observed for clinical signs at least once daily. Mice with tumors in excess of ˜2.5 g or with ulcerated tumors were euthanized, as were those found in obvious distress or in a moribund condition. All procedures carried out in this experiment were conducted in compliance with all the laws, regulations and guidelines of the NIH and with the approval of KARD Inc's Animal Care and Use Committee.

Treatment.

On day 13 after the implantation of ZR-75-1 tumor cells, the animals with tumors were randomized with similar size tumors across two groups. All animals weighed≧24 grams at the initiation of therapy. Mean group body weights at first treatment were well matched. All animals dosed with either the vehicle (PBS) or MUC1-Link-Trap at 10 mg/kg and injected intraperitonealy (IP) with a volume between 125 μl to 150 μl depending upon weight of the animal.

Measurements and Endpoints.

Tumor size was calculated from:

Tumor Volume (mm³)=l×w²/2

where w=width and l=length in mm of a ZR-75-1 tumor. Tumor weight was estimated with the assumption that 1 mg is equivalent to 1 mm³ of tumor volume.

Testing in this experiment was generally carried out adhering to the general Principles established by the groups of Schabel, Skipper, Griswold, Corbett, Leopold, Ross and the NCI (1-7), Body weights and tumor measurements were recorded daily 2.0 (some tunes every 3^{rd} day), Tumor burden (mg) was estimated from caliper measurements by the formula described above.

The primary endpoints used to evaluate efficacy were: tumor growth delay, tumor regression or complete cure, Treatment may cause partial regression (PR) or complete regression (CR) of the tumor in an animal. In a PR response, the tumor Size is 50% or less of it's size on day 1, but greater than zero, for three consecutive measurements during the course of the study. In a CR response, there is no measurable tumor mass for three consecutive measurements during the course of the study. The tumor is considered cured if there is no observable/palpable tumor for 60 days due to the treatment schedule.

Toxicity and Necropsy Examination.

The mice were examined frequently for overt signs of any adverse, drug-related side effects, in general, a drug study conducted on rodents, the females should be a minimum of 18 grams and males 19 grams, even though the general suggestion is higher body weight. The variation in body weight of all animals within the trial should be less than 5 grams. The animals are weighed daily during the drag treatment period including any weekends or holidays for at least 10 days or about 4 to 8 days after the last drug treatment, Weighing should be continued at least two time weekly for the entire study period. The daily weighing is usually the best method to assess toxicity in a flexible dose schedule screening trial.

Acceptable toxicity for cancer drugs in mice is defined by the NCI as group's mean body-weight loss of less than 20% during the test, and not more than one toxic death among ten related animals. Other toxicities encountered in primary screening trials: neurologic (stupor, ataxia, peripheral neuropathy, splay-foot-walk, seizures, coma, spasms, tremors, unconscious lying on its side and so forth.); respiratory problems; activity level (jumping, running, crouched, no movement, avoidance behavior); grooming or lack thereof, tissue damage, stomatitis; squealing; animals look poorly; and so on. There was no observable side effects of MUC1-Link-Trap in these mice.

Results.

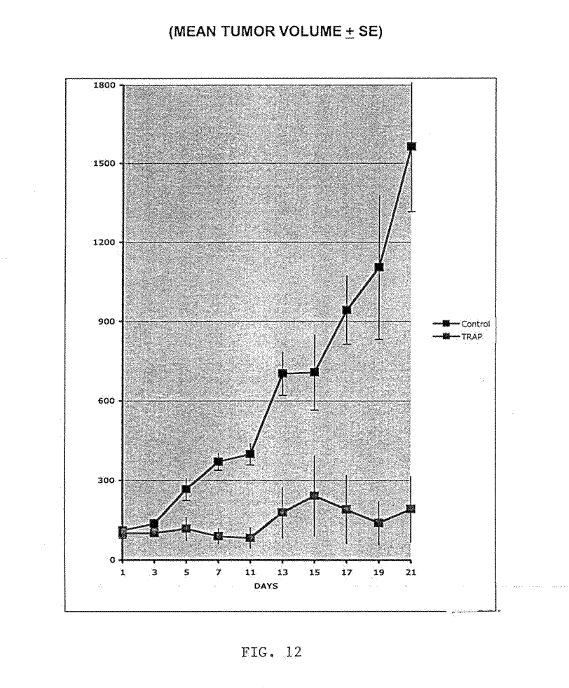

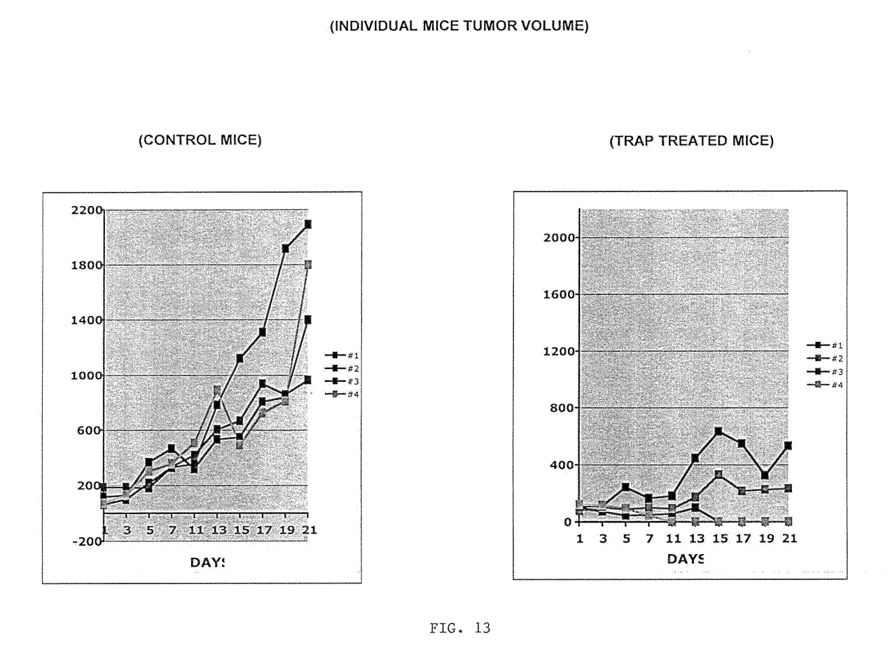

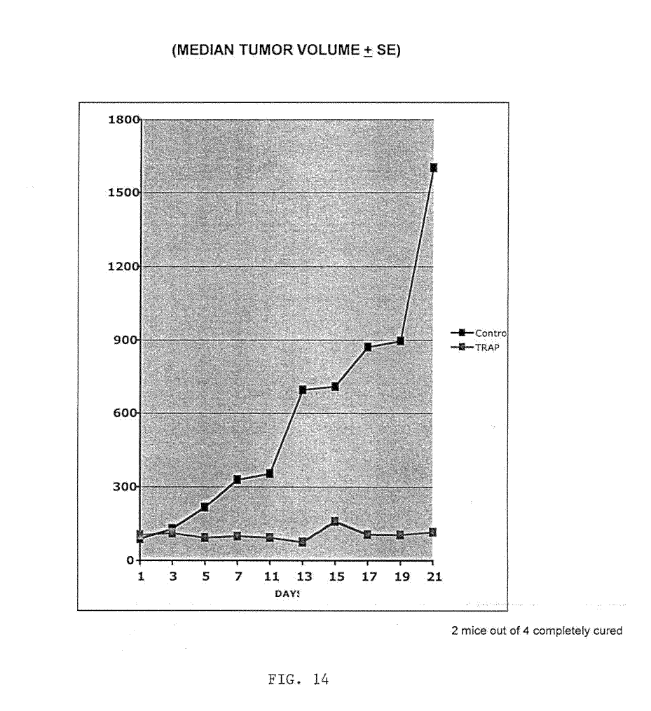

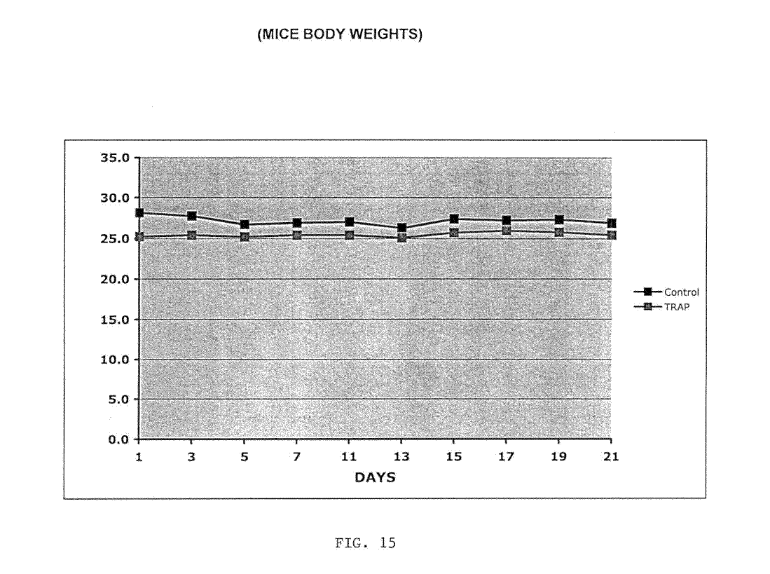

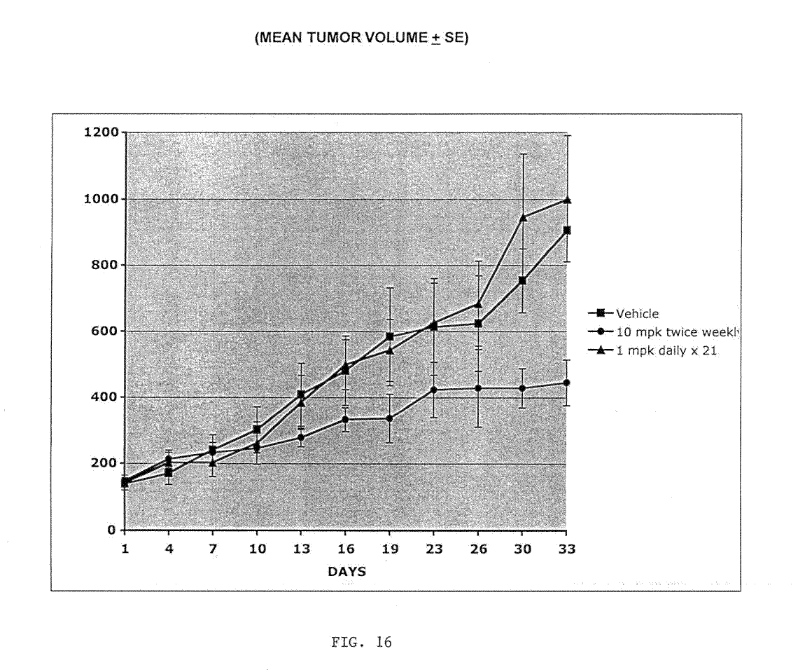

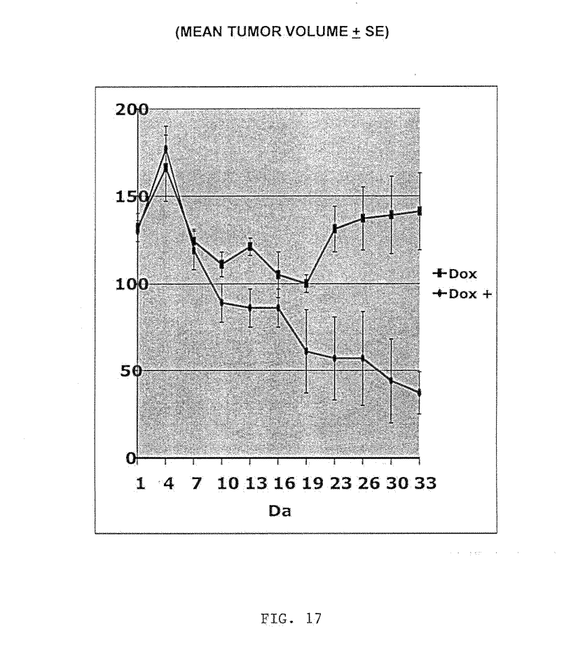

To assess antitumor activity of MUC1-Link-Trap, ZR-75-1 cells were implanted s.c. into the flanks of nude mice. Mice bearing tumors of ˜100 mm³ were treated with MUC1-Link-Trap at 10 mg/kg daily×21 days. Administration of MUC1-Link-Trap at 10 mg/kg/day×21 days significantly slowed growth compared with that obtained with vehicle (PBS) alone. Treatment with MUC1-Link-Trap as a single agent IP was well tolerated and statistically significant tumor growth delays were observed (FIGS. 12-15). In addition, administration of MUC1-Link-Trap at 10 mg/kg/day had no effect on body weight (data not shown). On day 11, one out of four mice in the study was completely tumor-free. On day 15, one additional mouse became completely tumor free. Significantly, tumors treated with MUC1-Link-Trap were no longer palpable by 60^{th} day in completely tumor-free mice. Tumors of the remaining mice in these groups were also significantly regressed and were remain below the starting tumor volumes. FIG. 16 shows distinct dosing regimens, while FIG. 17 shows an effective combination of the MUC1-Link-Trap with doxorubicin.

FIG. 18 shows coimmunoprecipitation of cell lysates expressing MUC1-C. MUC1-C was transiently expressed in wild-type CHO-K1 cells or the glycosylation-deficient Lec1 and Lec8 variants. Lysates were immunoblotted with anti-MUC1-C (left). The lysates were also incubated with GST-galectin-3 and the precipitates immunoblotted with anti-MUC1-C (right). Thus, N-linked glycosylation is necessary for binding to galectin-3.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference:
U.S. Pat. No. 4,683,202
U.S. Pat. No. 4,684,611
U.S. Pat. No. 4,879,236
U.S. Pat. No. 4,952,500
U.S. Pat. No. 5,217,879
U.S. Pat. No. 5,250,564
U.S. Pat. No. 5,264,419
U.S. Pat. No. 5,288,707
U.S. Pat. No. 5,302,523
U.S. Pat. No. 5,322,783
U.S. Pat. No. 5,384,253
U.S. Pat. No. 5,440,013
U.S. Pat. No. 5,446,128
U.S. Pat. No. 5,464,765
U.S. Pat. No. 5,475,085
U.S. Pat. No. 5,506,138
U.S. Pat. No. 5,538,877
U.S. Pat. No. 5,538,880
U.S. Pat. No. 5,550,251
U.S. Pat. No. 5,550,318
U.S. Pat. No. 5,552,534
U.S. Pat. No. 5,563,055
U.S. Pat. No. 5,580,859
U.S. Pat. No. 5,589,466
U.S. Pat. No. 5,610,042
U.S. Pat. No. 5,618,914
U.S. Pat. No. 5,656,610
U.S. Pat. No. 5,670,155
U.S. Pat. No. 5,670,488
U.S. Pat. No. 5,672,681
U.S. Pat. No. 5,674,976
U.S. Pat. No. 5,702,932
U.S. Pat. No. 5,710,245
U.S. Pat. No. 5,736,524
U.S. Pat. No. 5,739,018
U.S. Pat. No. 5,770,732
U.S. Pat. No. 5,780,448
U.S. Pat. No. 5,789,215
U.S. Pat. No. 5,801,148
U.S. Pat. No. 5,807,829
U.S. Pat. No. 5,811,387
U.S. Pat. No. 5,811,512
U.S. Pat. No. 5,811,515
U.S. Pat. No. 5,817,879
U.S. Pat. No. 5,824,544
U.S. Pat. No. 5,830,725
U.S. Pat. No. 5,840,833
U.S. Pat. No. 5,849,304
U.S. Pat. No. 5,851,826
U.S. Pat. No. 5,858,744
U.S. Pat. No. 5,859,184
U.S. Pat. No. 5,861,380
U.S. Pat. No. 5,869,455
U.S. Pat. No. 5,871,982
U.S. Pat. No. 5,871,983
U.S. Pat. No. 5,871,986
U.S. Pat. No. 5,877,578
U.S. Pat. No. 5,879,934
U.S. Pat. No. 5,888,502
U.S. Pat. No. 5,889,155
U.S. Pat. No. 5,925,565
U.S. Pat. No. 5,928,906
U.S. Pat. No. 5,939,237
U.S. Pat. No. 5,932,210
U.S. Pat. No. 5,935,819
U.S. Pat. No. 5,939,268
U.S. Pat. No. 5,945,100
U.S. Pat. No. 5,955,331
U.S. Pat. No. 5,965,695
U.S. Pat. No. 5,81,274
U.S. Pat. No. 5,994,136
U.S. Pat. No. 5,994,624
U.S. Pat. No. 6,013,516
U.S. Pat. No. 6,093,573
U.S. Pat. No. 6,117,974
U.S. Pat. No. 6,197,963
U.S. Pat. No. 6,261,569
U.S. Pat. No. 6,420,339
U.S. Pat. No. 6,436,697
U.S. Pat. No. 6,552,170
U.S. Pat. No. 6,566,493
U.S. Pat. No. 6,664,372
U.S. Pat. No. 6,706,862
U.S. Pat. No. 6,846,805
U.S. Pat. No. 6,946,542
U.S. Pat. No. 7,166,568
U.S. Pat. No. 7,202,332
U.S. Pat. No. 7,247,701
U.S. Pat. No. 7,587,286
U.S. Pat. No. 7,589,170
U.S. Pat. No. 7,610,156
U.S. Pat. No. 7,666,400
U.S. Pat. No. 7,705,012
U.S. Pat. No. 7,718,598
U.S. Pat. No. 7,863,239
U.S. Patent Appln. 2003/0187188
U.S. Patent Appln. 2005/0015232
U.S. Patent Appln. 2005/0043509
U.S. Patent Publn. 2010/012505
U.S. Patent Publn. 2011/0015138
U.S. Ser. No. 10/577,003
U.S. Ser. No. 13/026,858
U.S. Ser. No. 13/045,033
Abe and Kufe, Cancer Res., 49(11)2834-2839, 1989.
Ahmad et al., Nat Cell Biol., 9:1419-1427, 2007.
Almendro et al., J. Immunol., 157(12):5411-5421, 1996.
Amado and Chen, Science, 285(5428):674-676, 1999.
Angel et al., Cell, 49:729, 1987a.
Angel et al. Cell, 49:729, 1987b.
Armentano et al., Proc. Natl. Acad. Sci. USA, 87(16):6141-6145, 1990.
Atchison and Perry. Cell, 46:253, 1980.
Atchison and Perry, Cell, 48:121, 1987.
Ausuhel et al., Current Protocols in Molecular Biology, Greene Publishing Associates and Wiley Interscience, N.Y., 1994.
Baldus et al., Clin. Cancer Res., 10(8)2790-2796, 2004.
Banerji et al., Cell, 77:299, 1981.
Banerji et al. Cell, 33(3):729-740, 1981.
Bates, Mol. Biotechnol, 2(2):135-145, 1994.
Batra et al., Am. J. Respir. Cell Mol. Biol., 21(2):23-245, 1999,
Battraw and Hall, Theor. App. Genet., 82(2):161-168, 1991.
Berkhout et al., Cell, 59:273-282, 1989.
Bett et al., J. Virololgy, 67(10):5911-5921, 1993.
Bhattacharjee et al., J. Plant Bloch Biotech., 6(2):69-73, 1997.
Bilbao et al., FASEB J., 11(8):624-634, 1997.
Blackwell et al., Arch. Otolaryngol Head Neck Surg., 125(8)856-863, 1999.
Blanar et al., EMBO J., 8:1139, 1989.
Blomer et al., J. Virol., 71(9):6641-6649, 1997.
Bodanszky et al., J. Antibiot., 29(5):549-53, 1976.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1963.
Capaldi et al., Biochem. Biophys. Res. Comm., 74(2):425-433, 1977.
Caplen et al., Gene Ther., 6(3):454-459, 1999.
Carbonelli et al., FEMS Microbial. Lett., 177(1):75-82, 1999.
Case et al., Proc. Natl. Acad. Sci. USA, 96(6):2988-2993, 1999.
Celander and Haseltine, J. Virology, 61:269, 1987.
Celander et al., J. Virology, 62:1314, 1988.
Chandler et al., Proc. Natl. Acad. Sci. USA, 94(8):3590-601, 1997.
Chang et al., Mol. Cell. Biol., 9:2153, 1989.
Chatterjee et al., Proc. Natl. Acad. Sci. USA, 86:9114, 1989.
Chen and Okayama, Mol. Cell Biol., 7(8):2745-2752, 1987.
Chillon et al., J. Virol., 73(3):2537-2540, 1999.
Choi et al., J. Mol. Biol., 262(2):151-167, 1996.
Christou et al., Proc. Natl. Acad. Sci. USA, 84(12):3962-3966, 1987.
Clay et al., J. Immunol., 162:1749, 1999.
Cocea, Biotechniques, 23(5):814-816, 1997.
Coffey et al., Science, 282(5392):1332-1334, 1998.
Cohen et al. J. Cell. Physiol., 5:75, 1987.
Cohen et al., J. Med. Chem., 33:883-894, 1990.
Costa et al., Mol. Cell. Biol., 8:81-90, 1988.
Cripe et al., EMBO J., 6:3745, 1987.
Culotta and Hamer, Mol. Cell. Biol., 9:1376-1380, 1989.
D'Halluin et al., Plant Cell, 4(12):1495-1505, 1992.
Dandolo et al., J. Virology, 47:55-64, 1983.
DeLuca et al., J. Virol., 56(2):558-570, 1985.
Derby et al., Hear Res, 134(1-2):1-8, 1999.
Deschamps et al., Science, 230:1174-1177, 1985.
Dorai et al., Int. J. Cancer, 82(6):846-52, 1999.
Edbrooke et al., Mol. Cell. Biol., 9:1908-1916, 1989.
Edlund et al., Science, 230:912-916, 1985.
Engel and Kohn, Front Biosci, 4:e26-33, 1999. EPO 0273085
Fechheimer et al., Proc Natl. Acad. Sci. USA, 84:8463-8467, 1987.
Feldman et al., Cardiovasc. Res., 32(2):194-207, 1996.
Feldman et al., Semin. Interv. Cardiol., 1(3):203-208, 1996.
Feng and Holland, Nature, 334:6178, 1988.
Feng et al., Nat. Biotechnol., 15(9):866-870, 1997.
Firak and Subramanian, Mol. Cell. Biol., 6:3667, 1986.
Fisher et al., Hum. Gene Ther., 7(17):2079-2087, 1996.
Foecking and Hofstetter, Gene, 45(1):101-105, 1986.
Fraley et al., Proc. Natl. Acad. Sci. USA, 76:3348-3152, 1979.
Fujita et al., Cell, 49:357, 1987.
Fujiwara and Tanaka, Nippon Geka Gakkai Zasshi, 99(7):463-468, 1998.
Garoff and Li, Curr. Opin. Biotechnol., 9(5):464-469, 1998.
Garrido et al., J. Neurovirol., 5(3):280-288, 1999.
Gendler et al., J. Biol. Chem., 263:12820-12823, 1988.
Ghosh and Bachhawat, In: Liver Diseases, Targeted Diagnosis and Therapy Using Specific Receptors and Ligands, Wu et al., (Eds.), Marcel Dekker, NY, 87-104, 1991.
Gillies et al., Cell, 331717, 1983.
Gloss et al., EMBO J., 63735, 1987.
Gnant et al., Cancer Res., 59(14):3396-403, 1999.
Gnant et al., J. Natl. Cancer Inst., 91(20):1744-1750, 1999.
Godbout et al., Mol. Cell. Biol., 8:1169, 1988.
Goodbourn and Maniatis, Cell. 41(21:509-520, 1985.
Goodbourn et al., Cell, 45:601, 1986.
Gopal, Mol. Cell Biol., 5:1188-1190, 1985.
Graham and Prevec, Mol Biotechnol, 3(3):20′7-220, 1995.
Graham and Van Der Eb, Virology, 52:456-467, 1973.
Greene et al., Immunology Today, 10:272, 1989.
Gronenborn et al., Anal. Chem., 62(1):2-15, 1990.
Grosschedl and Baltimore, Cell, 41:885, 1985.
Haecker et al., Hum. Gene Ther., 7(15):1907-1914, 1996.
Han et al., J. Infect. Dis., 179:230-233, 1999.
Harland and Weintraub, J. Cell Biol., 101(3):1094-1099, 1983.
Haslinger and Karin, Proc. Natl. Acad. Sci. USA, 82:8572, 1985.
Hauber and Cullen, J. Virology, 62:673, 1988.
Hayashi et al., Neurosci. Lett., 267(1):37-40, 1999.
He et al., Plant Call Reports, 14 (2-3):192-196, 1994.
Hen et al., Nature, 321:249, 1986.
Hensel et al., Lymphokine Res., 8:347, 1989.
Hermens and Verhaagen, Prog. Neurobiol., 55(4):399-432, 1998.
Herr and Clarke, Cell, 45:461, 1986.
Hirochika et al., J. Virol., 61:2599, 1987.
Hirsch et al., Mol. Cell. Biol., 10:1959, 1990.
Holbrook et al., Virology, 157:211, 1987.
Holzer et al. Virology, 253(1):107-114, 1999.
Horlich and Benfield, Mol. Cell. Biol., 9:7396, 1989.
Holt and Lin, Plant Physiology, 111:166, 1996.
Howard et al. Ann. NY Acad. Sci., 880:352-365, 1999.
Huang et al., Cancer Biol Ther., 2:702-706, 2003.
Huang et al., Cancer Res., 65:10413-10422, 2005.
Huang et al., Cell, 27:245, 1981.
Huard et al., Neuromuscul Disord, 7(5):299-313, 1997.
Hug et al., Mol. Cell. Biol., 83065-3079, 1988.
Hwang et al., Mol. Cell. Biol., 10:535, 1990.
Imagawa et al., Cell, 51:251, 1987.
Imai et al. Nephrologie, 19(7):397-402, 199.
Imbra and Karin, Nature, 323:555, 1986.
Imler et al., Mol. Cell. Biol., 7:2558, 1987.
Imperiale and Nevins, Mol. Cell. Biol., 4:875, 1984.
Irie et al., Antisense Nucleic Acid Drug Dev., 9(4):341-349, 1999.
Jackson, Seminars in Oncology, 24:L164-172, 1997.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., In: Biotechnology And Pharmacy, Pezzuto et al. (Eds.), Chapman and Hall, New York, 1993.
Johnson et al., Mol. Cell. Biol., 9(8):3393-1399, 1989.
Johnston et al., J. Virol., 73(6):4991-5000, 1999.
Jones et al., Br. J. Pharmacol., 145(8):1093-109, 2005.
Kadeseh and Berg, Mol. Cell. Biol., 6:2593, 1986.
Kaeppler et al., Plant Cell Rep., 8:415-418, 1990.
Kaneda et al., Science, 243:375-373, 1989.
Karin et al., Mol. Cell. Biol., 7:606, 1987.
Karin et al., Mol. Cell. Biol., 7:606, 1987.
Katinka et al., Cell, 20:393.1980.
Katinka et al., Nature, 290:720, 1981.
Kato et al., J. Biol. Chem., 266:3361-3364, 1991.
Kaufman et al., Arch. Ophthalmol., 117(7):925-928, 1999.
Kawamoto et al., Mol. Cell. Biol., 8:267, 1988.
Kay, Haemophilia, 4(4):389-199, 1998.
Kiledjian et al. Mol. Cell. Biol., 8:145, 1988.
Klamut et al., Mol. Cell. Biol., 10:193, 1990.
Klimatcheva et al., Front Biosci 4:D481-496, 1999.
Koch et al., Mol. Cell. Biol., 9:303, 1989.
Kohut et al., Am. J. Physiol., 275(6Pt1):L1089-1094, 1998.
Kooby et al., FASEB J, 13(11):1325-34, 1999.
Kraus et al. FEBS Lett., 428(3):165-170, 1998.
Kriegler and Botchan, Mol. Cell. Biol., 3:325, 1983.
Kriegler et al., Cell, 38:483, 1984a.
Kriegler et al., In: Cancer Cells 2/Oncogenes and Viral Genes, Van de Woude et al. eds. Cold Spring Harbor, Cold Spring harbor Laboratory, 1984b.
Krisky et al., Gene Ther, 5(11):1517-1530, 1998a.
Krisky et al., Gene Ther, 5(12):1593-1603, 1998b.
Kuhl et al., Hybridoma, 3:223-232, 1984.
Kuhl et al., Cell, 50:1057, 1987.
Kunz et al., Nucl. Acids Res., 17:1121, 1989.
Lachmann and Efstathiou, Curr. Opin. Mol. Ther., 1(5):622-632, 1999.
Lareyre et al., J. Biol. Chem., 274(12):8282-8290, 1999.
Larsen et al., Proc. Natl. Acad. Sci. USA, 83:8283, 1986.
Laspia et al., Cell, 59:283, 1989.
Latimer et al., Mol. Cell, Biol., 10:760, 1990.
Lazzeri, Methods Mol. Biol., 49:95-106, 1995.
Lee et al., Environ. Mutagen., 13(1):54-59, 1989.
Lee et al., Nature, 294:278, 1981.
Lee et al., Nucleic Acids Res., 12:4191-706, 1984.
Leibowitz et al., Diabetes, 48(4):745-753, 1999.
Leng et al., J. Biol. Chem., 232:19321-19330, 2007.
Lesch, Biol Psychiatry, 45(3):247-253, 1999.
Levenson et al., Hum. Gene Ther., 9(8):1.233-1236, 1998.
Levitan et al., J. Biol. Chem., 280:33374-33386, 2005.
Li et al., Cancer Biol. Ther., 2:18′7-191, 2003b.
Li et al., J. Biol. Chem., 276:35239-35242, 2001.
Li et al., J. Biol. Chem., 276:6061-6064, 2001.
Li et al., Mol. Cancer Res., 1:765-775, 2003c.
Li et al., Mol. Cell Biol., 18:7216-7224, 1993.
Li et al., Oncogene, 22:6107-6110, 2003a.
Ligtenberg et al., J. Biol. Chem., 267, 6171-6177, 1992.
Lin et al., Mol. Cell. Biol., 10:850, 1990.
Lundstrom, J. Recept Signal Transduct. Res., 19(1-4):673-686, 1999.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macao, Nat. Struct. Mol. Biol., 13, 71-76, 2006.
Macejak and Sarnow, Nature, 353:90-94, 1991.
Majors and, Varmus, Proc. Natl. Acad. Sci. USA, 80:5866, 1983.
Marienfeld et al., Gene Ther., 6(6):1101-1113, 1999.
Mastrangelo et al., Biotechnol. Bioeng., 65(3):298-305, 1999.
MeNeall et al., Gene, 76:81, 1989.
Merlo et al., Cancer Res., 49, 6966-6971, 1989.
Merrifield, J. Am. Chem. Soc., 85:2149-2154, 1963.
Miksicek et al., Cell, 46:203, 1986.
Miller et al., J. Pharmacol. Exp. Ther., 264:11-16, 1993.
Miyatake et al., Gene Ther., 6:564-572, 1999.
Moldawer et al., Shock, 12(2):83-101, 1999.
Mordacq and Linzer, Genes and Dev., 3:760, 1989.
Moreau et al., Nucl. Acids Res., 9:6047, 1981.
Moriuchi et al., Cancer Res, 58(24):5731-5737, 1998.
Morrison et al., J. Gen. Virol., 78(Pt 4):873-878, 1997.
Muesing et al., Cell, 48:691, 1987.
Naldini et al., Science, 272(5259):263-267, 1996.
Navia et al., Curr. Opin. Struct. Biol., 2:202-210, 1997.
Neuberger et al., Nucleic Acids Res., 16(14B):6713-6724, 1988.
Neumann et al., Proc. Natl. Acad. Sci. USA, 96(16):9345-9350, 1999.
Ng et al., Nuc. Acids Res., 17:601, 1989.
Nicolau and Sene, Biochim, Biophys. Acta, 721:185-190, 1982.
Nicolau et al., Methods Enzymol., 149:157-176, 1987.
Nomoto et al., Gene, 236(2):759-271, 1999.
Omirulleh et al., Plant Mol. Biol., 21(3):415-428, 1993.
Omitz et al., Mol. Cell. Biol., 7:3466, 1987.
Ondek et al. EMBO J., 6:1017, 1987.
Palmiter et al., Cell, 29:701, 1982.
Parks et al., J. Virol., 71(4)3293-8, 1997.
PCT Appln. WO 92/17598
PCT Appln. WO 94/09699
PCT Appln. WO 95/06128
PCT Appln. PCT/US00/14667
PCT Appln. PCT/US99/11913
PCT Appln. PCT/US99/18441
Pech et al., Mol. Cell. Biol., 9:396, 1989.
Pelletier and Sonenberg, Nature, 334(6180):320-325, 1988.
Peptide Synthesis, 1985
Perales et al., Proc. Natl. Acad. Sci. USA, 91:4086-4090, 1994.
Percy et al., Cancer Res., 52(22):6365-6370, 1992.
Perez-Stable and Constantini, Mol. Cell. Biol., 10:1116, 1990.
Petrof, Eur Respir. J, 11(2):492-497, 1998.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Potrykus et al., Mol. Gen. Genet., 199(2):169-177, 1985.
Potter et al., Proc. Natl. Acad. Sci. USA, 81:7161-7165, 1984.
Protective Groups in Organic Chemistry, 1973
Protein NMR Spectroscopy, Principles and Practice, Cavanagh et al., Academic Press. San Diego, 1996.
Queen and Baltimore, Cell, 35:741, 1983.
Quinn et al., Mol. Cell. Biol., 9:4713, 1989.
Rabinovitch et al., Diabetes, 48(6):1223-1229, 1999.
Raina et al., EMBO J., 25:3774-3783, 2006.
Raina et al., J. Biol. Chem., 279:20607-20612, 2004.
Reddy et al., Virology, 251(2):414-26, 1998.
Redondo et al., Science, 247:1225, 1990.
Reisman and Roger, Mol. Cell. Biol., 9:3571, 1989.
Remington's Pharmaceutical Sciences, 15^{th} Ed., 1035-1038 and 1570-1580, 1990.
Remington's Pharmaceutical Sciences, 15^{th} Ed., 33:624-652, 1990.
Ren et al., Cancer Cell, 5:163-175, 2004.
Ren et al., J. Biol. Chem., 277:17616-17622, 2002.
Resendez Jr. et al., Mol. Cell. Biol., 8:4579, 1988.
Rhodes et al., Methods Mol. Biol., 55:121-131, 1995.
Rippe et al., Mol. Cell. Biol., 9(5):2224-22277, 1989.
Rippe, et al., Mol. Cell. Biol., 10:689-695, 1990.
Rittling et al., Nucl. Acids Res., 1′7:1619, 1989.
Robbins and Ghivizzani, Pharmacol Ther, 80(1):35-47, 1998.
Robbins et al., Trends Biotechnol., 16(1):35-40, 1998.
Sambrook et al., In: Molecular cloning: a laboratory manual, 2^{nd} Ed., Cold Spring Harbor Laboratory Press, Cold Spring Harbor, N.Y., 1989.
Sawai et al., Mol. Genet. Metab., 67(1):36-42, 1999.
Schaffner et al., J. Mol. Biol., 201:81, 1988.
Schroeder et al., Oncogene, 23:5739-5747, 2004.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Siddiqui et al., Proc. Natl. Acad. Sci. USA, 85:2320-2323, 1988.
Sleigh and Lockett, J. EMBO, 4:3831, 1985.
Smith et al., Neuron., 20:1093-1102, 1998.
Solid Phase Peptide Synthelia, 1984
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Stewart et al., Arch. Biochem. Biophys. 365:71-74; 1999.
Stuart et al., Nature, 317:828, 1985.
Sullivan and Peterlin, Mol. Cell. Biol., 7:3315, 1987.
Suzuki et al., Blochem Biophys Res Common, 252(3):686-90, 1998.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J. Virology, 62:614, 1988.
Timiryasova et al., Int. J. Oncol., 14(5):845-854, 1999.
Treisman, Cell, 42:889, 1985.
Tronche et al. Mol. Biol. Med., 7:173, 1990.
Tronche et al., Mol. Cell. Biol., 9:4759, 1989.
Trudel and Constantini. Genes and Dev., 6:954, 1987.
Truscott et al., J. Cell Biol., 163(4):707-713, 2003.
Tsukada et al., Plant cell Physiol., 30(4)599-604, 1989.
Tsumaki et al., J. Biol. Chem., 273(36):22861-22864, 1998.
Tur-Kaspa et al., Mol. Cell Biol., 6:716-718, 1986.
Tyndall et al., Nuc. Acids. Res., 9:6231, 1981.
Vanderkwaak et al., Gynecol Oncol. 74(2):227-234, 1999.
Vasseur et al., Proc. Natl. Acad. Sci. USA, 77:1068, 1980.
Vermeer et al., Nature, 422(6929):322-6, 2003.
Wagner et al., Proc. Natl. Acad. Sci. USA 87(9):3410-3414, 1990.
Wang and Calame, Cell, 47:241, 1986.
Wang et al., Infect. Immun., 66:4193-202, 1998.
Weber et al., Cell, 36:983, 1984.
Wei et al., Cancer Cell, 7:167-178, 2005.
Weihl et al., Neurosurgery, 44(7):239-252, 1999.
Wen et al., J. Biol. Chem., 278:38029-38039, 2003.
White et al. J. Virol., 73(4):2832-2840, 1999.
Wider, BioTechniques, 29:1278-1.294, 2000.
Wilson, J. Clin. Invest. 98(11):2435, 1996.
Winoto and Baltimore, Cell, 59:649, 1989.
Wang et al., Gene, 10:87-94, 1980.
Wu and Wu, Adv. Drug Delivery Rev., 12:159-167, 1993.
Wu and Wu, J. Biol. Chem., 262:4429-4432, 1987.
Wu et al., Biochem. Biophys. Res. Commun., 233(1):271-216, 1997.
Wu et ed., Cancer Res., 58(8): 1605-8, 1998.
Yamada et al., Brain Res., 833(2):302-307, 1999.
Yamamoto et al., J. Biol. Chem., 272:12492-12494, 1997.
Yeung et al., Gene Ther., 6(9):1536-1544, 1999.
Yin et al., J. Biol. Chem., 278:35458-35464, 2003.
Yin et al., J. Biol. Chem., 279:45721-45727, 2004.
Yin et al., J. Biol. Chem., 282:257-266, 2007.
Yowl et al., J. Gastrointest. Surg., 3(1):34-48, 1999.
Young et al., Cell. 112(1):41-50, 2003.
Yutzey et al. Mol. Cell. Biol., 9:1397, 1989.
Zhao-Emonet et al., Biochim. Biophys. Acta. 1442(2-3):109-119, 1998.
Zheng et al., J. Gen. Virol., 80(Pt 7):1735-1742, 1999.
Zhou et al., Nature, 361(6412):543-547, 1993.
Zufferey et al., Nat. Biotechnol., 15(9):871-875, 1997.

1. A method of inhibiting a MUC1-positive cancer cell comprising contacting said cell with a MUC1 ligand trap, said ligand trap comprising:
(a) a first MUC1 segment comprising least a portion of a MUC1 external domain (ED);
(b) at least a portion of an immunoglobulin Fc domain; and either or both of
(c) a first linker disposed between (a) and (b) and/or glycosylation of residue Asn36 of the MUC1 ECD amino acid sequence.

2. The method of claim 1, wherein said first MUC1 segment comprises at least 50 residues.

3. The method of claim 1, wherein said first MUC1 segment lacks tandem repeats.

4. The method of claim 3, wherein said first MUC1 segment comprises the MUC1 SEA domain.

5. The method of claim 1, wherein said Fc domain portion comprises a constant region from IgG1 or IgG2a.

6. The method of claim 1, said cancer cell is a solid tumor cell.

7. The method of claim 6, wherein said solid tumor cell is a lung cancer cell, a brain cancer cell, a head & neck cancer cell, a breast cancer cell, a skin cancer cell, a liver cancer cell, a pancreatic cancer cell, a stomach cancer cell, a colon cancer cell, a rectal cancer cell, a uterine cancer cell, a cervical cancer cell, an ovarian cancer cell, a testicular cancer cell, a skin cancer cell or a esophageal cancer cell.

8. The method of claim 1, where said cancer cell is a leukemia or myeloma cell.

9-11. (canceled)

12. The method of claim 1, wherein said first linker comprises the sequence GGGGSGGGGSGGGGS (SEQ ID NO:10).

13. (canceled)

14. The method of claim 1, further comprising contacting said cancer cell with a second anti-cancer agent.

15-20. (canceled)

21. The method of claim 1, wherein the ligand trap comprises a second MUC1 segment comprising at least a portion of a MUC1 ED.

22-27. (canceled)

28. A method of inhibiting a MUC1-positive cancer in a subject comprising administering to said subject a MUC1 ligand trap, said ligand trap comprising:
(a) a first MUC1 segment comprising at least a portion of a MUC1 external domain (ED);
(b) at least a portion of an immunoglobulin Fc domain; and either or both of
(c) a first linker disposed between (a) and (b) and/or glycosylation of residue Asn36 of the MUC1 ECD amino acid sequence.

29. The method of claim 28, wherein said first MUC1 segment comprises at least 15 residues.

30. The method of claim 28, wherein said first MUC1 segment lacks tandem repeats.

31. The method of claim 30, wherein said first MUC1 segment comprises the MUC1 SEA domain.

32. The method of claim 28, wherein said Fc domain portion comprises an IgG1 or IgG2a constant region.

33. The method of claim 28, said cancer is a solid tumor.

34. The method of claim 33, wherein said solid tumor is a lung cancer, a brain cancer, a head & neck cancer, a breast cancer, a skin cancer, a liver cancer, a pancreatic cancer, a stomach cancer, a colon cancer, a rectal cancer, a uterine cancer, a cervical cancer, an ovarian cancer, a testicular cancer, a skin cancer or a esophageal cancer.

35. The method of claim 28, where said cancer is a leukemia or myeloma.

36-38. (canceled)

39. The method of claim 28, wherein said first linker comprises the sequence GGGGSGGGGSGGGGS (SEQ ID NO:10).

40. (canceled)

41. The method of claim 28, further comprising providing to said subject a second anti-cancer therapy.

42-47. (canceled)

48. The method of claim 28, wherein administering comprises intravenous, intra-arterial, oral, intratumoral, subcutaneous, topical or intraperitoneal administration.

49. The method of claim 28, wherein administering comprises local, regional, systemic, or continual administration.

50. The method of claim 28, wherein the ligand trap comprises a second MUC1 segment comprising at least a portion of a MUC1 ED.

51-56. (canceled)

57. A pharmaceutical composition comprising a MUC1 ligand trap, said ligand trap comprising:
(a) a first MUC1 segment comprising at least a portion of a MUC1 external domain (ED);
(b) at least a portion of an immunoglobulin Fc domain; and either or both of
(c) a first linker disposed between (a) and (b) and/or glycosylation of residue Asn36 of the MUC1 ECD amino acid sequence.

58. The pharmaceutical composition of claim 57, wherein said first MUC1 segment comprises at least 15 residues.

59. The pharmaceutical composition of claim 57, wherein said first MUC1 segment lacks tandem repeats.

60. The pharmaceutical composition of claim 57, wherein said first MUC1 segment comprises the MUC1 SEA domain.

61. The pharmaceutical composition of claim 57, wherein said Fc domain portion comprises an IgG1 or IgG2a constant region.

62. (canceled)

63. The pharmaceutical composition of claim 57, wherein said first linker comprises the sequence GGGGSGGGGSGGGGS (SEQ ID NO:10) and/or said glycosylation is an N-linked glycan comprising beta-galactosides.

64. The pharmaceutical composition of claim 57, wherein said ligand trap is encapsulated or embedded in a delivery vehicle.

65. The pharmaceutical composition of claim 64, wherein said delivery vehicle is a liposome, a lysosome, a microcapsule or a nanoparticle.

66. The pharmaceutical composition of claim 57, wherein N- and C-termini of the linker are (i) L and AAA, respectively, on a flexible linker and (ii) LEA and AAA on a helical linker.

67. (canceled)

68. The pharmaceutical composition of claim 57, wherein the ligand trap comprises a second MUC1 segment comprising at least a portion of a MUC1 ED.

69-73. (canceled)
